SEQUENCE LISTING (20 sequences)

SEQ ID NO: 1 - DNA, 750 bp, Drosophila melanogaster
  CDS (1)..(750)
atggcggaggcagcatcctgtgcccgaaaggggaccaagtacgccgagggcacccagcccttcaccgtcctcatcgagggcaacatcggcagcgggaagaccacgtatttgaaccacttcgagaagtacaagaacgacatttgcctgctgaccgagcccgtcgagaagtggcgcaacgtcaacggggtaaatctgctggagctgatgtacaaagatcccaagaagtgggccatgccctttcagagttatgtcacgctgaccatgctgcagtcgcacaccgccccaaccaacaagaagctaaaaataatggagcgctccatttttagcgctcgctattgcttcgtggagaacatgcgacgaaacggctcgctggagcagggcatgtacaatacgctggaggagtggtacaagttcatcgaagagtccattcacgtgcaggcggacctcatcatatatctgcgcacctcgccggaggtggcgtacgaacgcatccggcagcgggctcgttctgaggagagctgcgtgccgcttaagtaccttcaggagctgcatgagttgcacgaggactggttgatacaccagagacgaccgcagtcgtgcaaggtcctagtcctcgatgccgatctgaacctggaaaacattggcaccgagtaccagcgctcggagagcagcatattcgacgccatctcaagtaaccaacagccctcgccggttctggtgtcgcccagcaagcgccagagggtcgccaga

SEQ ID NO: 2 - protein, 250 aa, Drosophila melanogaster
MAEAASCARKGTKYAEGTQPFTVLIEGNIGSGKTTYLNHFEKYKNDICLLTEPVEKWRNVNGVNLLELMYKDPKKWAMPFQSYVTLTMLQSHTAPTNKKLKIMERSIFSARYCFVENMRRNGSLEQGMYNTLEEWYKFIEESIHVQADLIIYLRTSPEVAYERIRQRARSEESCVPLKYLQELHELHEDWLIHQRRPQSCKVLVLDADLNLENIGTEYQRSESSIFDAISSNQQPSPVLVSPSKRQRVAR

SEQ ID NO: 3 - DNA, 747 bp, Bombyx mori
  CDS (1)..(747)
atgagtgccaacaatgttaaaccattcaccgtgttcgtggaaggtaacataggtagcggtaaaacaacatttctggaacattttcgtcagtttgaggatatcactttgttgacggagcccgttgaaatgtggcgagatcttaaaggttgcaatcttttggaactcatgtacaaagatccagaaaaatgggcgatgacattccagtcatacgtttccttgacgatgttggacatgcaccggagacctgctccaactccagtaaagctaatggagcgatcattattcagtgcgagatactgcttcgttgaacacattatgagaaataatacactccatccagcacagtttgcagtacttgatgagtggttccgattcatccaacacaacattcctattgatgctgatttgatagtatatctaaagacatcaccttcaatagtgtaccaaaggataaaaaagagagctcgttcagaagagcagtgtgtgcccctgtcatacattgaggaactgcataggttgcatgaggactggctaatcaacaggatacatgctgaatgtcccgcaccagtattagtgttagatgctgatttagacctctctcagataaccgatgaatacaagagaagtgagcatcaaattttaagaaaggctgttaatgtagttatgagttcaccaaacaagcatagcccaaagaaaccaatatcaacaacaccaatcaagatcacacctcacatgaggatcttataa

SEQ ID NO: 4 - protein, 248 aa, Bombyx mori
MSANNVKPFTVFVEGNIGSGKTTFLEHFRQFEDITLLTEPVEMWRDLKGCNLLELMYKDPEKWAMTFQSYVSLTMLDMHRRPAPTPVKLMERSLFSARYCFVEHIMRNNTLHPAQFAVLDEWFRFIQHNIPIDADLIVYLKTSPSIVYQRIKKRARSEEQCVPLSYIEELHRLHEDWLINRIHAECPAPVLVLDADLDLSQITDEYKRSEHQILRKAVNVVMSSPNKHSPKKPISTTPIKITPHMRIL

SEQ ID NO: 5 - DNA, 837 bp, Xenopus laevis
  CDS (1)..(837)
atgtcagtactactagctgcccggacatgtatacggctctgttgtactgaacataaaacaggcgctctggcgaggtttaacctcggcgcaaatactgccctgactgttagaagaatagcgagcgctttgtgcggcagatgcaacatcatgcggagaggaatattgccctcggggagcacaggtaatggtctaaaaagccgagagaagagcacagtaatttgtgtggaggggaatattgcaagtggaaaaacaagctgcttggattatttttctaatactccagatcttgaggtattcaaggagcctgtagctaaatggagaaatgtctgtggccataacccacttggtttaatgtatcaagatcctaacaagtggggcttaactttgcagacgtacgtgcaactcaccatgttggacattcacaccaaaccatcaatctcgcctgttaaaatgatggaaaggtcaatttacagtgcaaagtatatctttgtagagaacttgtatcagagcggaaaaatgccagccgtggattatgccattttaacagagtggtttaaatggattgtaaagaacaccgatacctcggttgatctgatcgtttatctgcagacatctccagaaatctgttaccagagactaaagaagaggtgtagagaagaggagagtgttataccactggaatatctgtgtgcaatccacaatctctatgaagactggctagttaaacagacgtccttctcagtgccggcacccgtgcttgtaatagatggtaataaagaactagaggaattgactcaacactatgaagagaatcgcaccagcatcttgtcactgtaa

SEQ ID NO: 6 - protein, 278 aa, Xenopus laevis
MSVLLAARTCIRLCCTEHKTGALARFNLGANTALTVRRIASALCGRCNIMRRGILPSGSTGNGLKSREKSTVICVEGNIASGKTSCLDYFSNTPDLEVFKEPVAKWRNVCGHNPLGLMYQDPNKWGLTLQTYVQLTMLDIHTKPSISPVKMMERSIYSAKYIFVENLYQSGKMPAVDYAILTEWFKWIVKNTDTSVDLIVYLQTSPEICYQRLKKRCREEESVIPLEYLCAIHNLYEDWLVKQTSFSVPAPVLVIDGNKELEELTQHYEENRTSILSL

SEQ ID NO: 7 - DNA, 28 bp, Artificial Sequence
  Description of Artificial Sequence PCR primer sequence
cgcggatccatggcggaggcagcatcct

SEQ ID NO: 8 - DNA, 30 bp, Artificial Sequence
  Description of Artificial Sequence PCR primer sequence
cggaattcttatctggcgaccctctggcgt

SEQ ID NO: 9 - DNA, 711 bp, hybrid
  CDS (1)..(711): 1-270 Bombyx mori; 271-711 Drosophila melanogaster.
atgagtgccaacaatgttaaaccattcaccgtgttcgtggaaggtaacataggtagcggtaaaacaacatttctggaacattttcgtcagtttgaggatatcactttgttgacggagcccgttgaaatgtggcgagatcttaaaggttgcaatcttttggaactcatgtacaaagatccagaaaaatgggcgatgacattccagtcatacgtttccttgacgatgttggacatgcaccggagacctgctccaactccagtaaagctaatggagcgctccatttttagcgctcgctattgcttcgtggagaacatgcgacgaaacggctcgctggagcagggcatgtacaatacgctggaggagtggtacaagttcatcgaagagtccattcacgtgcaggcggacctcatcatatatctgcgcacctcgccggaggtggcgtacgaacgcatccggcagcgggctcgttctgaggagagctgcgtgccgcttaagtaccttcaggagctgcatgagttgcacgaggactggttgatacaccagagacgaccgcagtcgtgcaaggtcctagtcctcgatgccgatctgaacctggaaaacattggcaccgagtaccagcgctcggagagcagcatattcgacgccatctcaagtaaccaacagccctcgccggttctggtgtcgcccagcaagcgccagagggtcgccaga

SEQ ID NO: 10 - protein, 237 aa, hybrid
MSANNVKPFTVFVEGNIGSGKTTFLEHFRQFEDITLLTEPVEMWRDLKGCNLLELMYKDPEKWAMTFQSYVSLTMLDMHRRPAPTPVKLMERSIFSARYCFVENMRRNGSLEQGMYNTLEEWYKFIEESIHVQADLIIYLRTSPEVAYERIRQRARSEESCVPLKYLQELHELHEDWLIHQRRPQSCKVLVLDADLNLENIGTEYQRSESSIFDAISSNQQPSPVLVSPSKRQRVAR

SEQ ID NO: 11 - DNA, 786 bp, hybrid
  CDS (1)..(786): 1-309 Drosophila melanogaster; 310-786 Bombyx mori.
atggcggaggcagcatcctgtgcccgaaaggggaccaagtacgccgagggcacccagcccttcaccgtcctcatcgagggcaacatcggcagcgggaagaccacgtatttgaaccacttcgagaagtacaagaacgacatttgcctgctgaccgagcccgtcgagaagtggcgcaacgtcaacggggtaaatctgctggagctgatgtacaaagatcccaagaagtgggccatgccctttcagagttatgtcacgctgaccatgctgcagtcgcacaccgccccaaccaacaagaagctaaaaataatggagcgatcattattcagtgcgagatactgcttcgttgaacacattatgagaaataatacactccatccagcacagtttgcagtacttgatgagtggttccgattcatccaacacaacattcctattgatgctgatttgatagtatatctaaagacatcaccttcaatagtgtaccaaaggataaaaaagagagctcgttcagaagagcagtgtgtgcccctgtcatacattgaggaactgcataggttgcatgaggactggctaatcaacaggatacatgctgaatgtcccgcaccagtattagtgttagatgctgatttagacctctctcagataaccgatgaatacaagagaagtgagcatcaaattttaagaaaggctgttaatgtagttatgagttcaccaaacaagcatagcccaaagaaaccaatatcaacaacaccaatcaagatcacacctcacatgaggatcttataa

SEQ ID NO: 12 - protein, 261 aa, hybrid
MAEAASCARKGTKYAEGTQPFTVLIEGNIGSGKTTYLNHFEKYKNDICLLTEPVEKWRNVNGVNLLELMYKDPKKWAMPFQSYVTLTMLQSHTAPTNKKLKIMERSLFSARYCFVEHIMRNNTLHPAQFAVLDEWFRFIQHNIPIDADLIVYLKTSPSIVYQRIKKRARSEEQCVPLSYIEELHRLHEDWLINRIHAECPAPVLVLDADLDLSQITDEYKRSEHQILRKAVNVVMSSPNKHSPKKPISTTPIKITPHMRIL

SEQ ID NO: 13 - DNA, 20 bp, Artificial Sequence
  Description of Artificial Sequence PCR primer sequence
acgtttggtggtggcgacca

SEQ ID NO: 14 - DNA, 20 bp, Artificial Sequence
  Description of Artificial Sequence PCR primer sequence
ctccgggagctgcatgtgtc

SEQ ID NO: 15 - DNA, 38 bp, Artificial Sequence
  Description of Artificial Sequence PCR primer sequence
ctaaaaatggagcgctccattagctttactggagttgg

SEQ ID NO: 16 - DNA, 35 bp, Artificial Sequence
  Description of Artificial Sequence PCR primer sequence
ccagtaaagctaatggagcgctccatttttagcgc

SEQ ID NO: 17 - DNA, 35 bp, Artificial Sequence
  Description of Artificial Sequence PCR primer sequence
gaataatgatcgctccattatttttagcttcttgt

SEQ ID NO: 18 - DNA, 35 bp, Artificial Sequence
  Description of Artificial Sequence PCR primer sequence
aagctaaaaataatggagcgatcattattcagtgc

SEQ ID NO: 19 - DNA, 42 bp, Artificial Sequence
  Description of Artificial Sequence PCR primer sequence
tatcgcggatccatgagtgccaacaatgttaaaccattcacc

SEQ ID NO: 20 - DNA, 46 bp, Artificial Sequence
  Description of Artificial Sequence PCR primer sequence
ccggaattcgtcgacttataagatcctcatgtgaggtgtgatcttg

(19) United States
(12) Patent Application Publication
(10) Pub. No.: US 2004/0072168 A1
(43) Pub. Date: Apr. 15, 2004

(54) Novel deoxynucleoside kinase enzyme variants

(76) Inventors: Wolfgang Knecht, Goteborg (SE); Birgitte Minch-Petersen, Farum (DK); Jure Piskur, Kobenhavn (DK)

Correspondence Address: BIRCH STEWART KOLASCH & BIRCH, PO BOX 747, FALLS CHURCH, VA, 22040-0747, US

(21) Appl. No.: 10/275,879

(22) PCT Filed: May 7, 2001
(86) PCT No.: PCT/DK01/00316

(30) Foreign Application Priority Data
May 12, 2000 (DK) PA200000781

(51) Int. Cl. A61K 48/00; C12Q 1/68; C07H 21/04; C12N 9/12; C12P 21/02; C12N 1/21

(52) U.S. Cl. 435/6; 435/69.1; 435/194; 435/252.3; 435/235.1; 435/320.1; 514/44; 536/23.2

(57) ABSTRACT

This invention relates to novel multi-substrate deoxyribonucleoside kinase variants. More specifically the invention provides novel deoxyribonucleoside kinase variants derived from insects or lower vertebrates, in particular from Drosophila melanogaster, from Bombyx mori, or from Xenopus laevis, novel polynucleotides encoding multi-substrate nucleoside kinase variants, vector constructs comprising the polynucleotide, host cells carrying the polynucleotide or vector, methods of sensitising cells to prodrugs, method of inhibiting pathogenic agents in warm-blooded animals, and pharmaceutical compositions comprising deoxyribonucleoside kinase variants of the invention.

### TECHNICAL FIELD

This invention relates to hovel multi-substrate deoxyribonucleoside kinase variants. More specifically the invention provides novel deoxyribonucleoside kinase variants derived from insects or lower vertebrates, in particular from Drosophila melanogaster, from Bombyx mori, or from Xenopus laevis, novel polynucleotides encoding multi-substrate nucleoside kinase variants, vector constructs comprising the polynucleotide, host cells carrying the polynucleotide or vector, methods of sensitising cells to prodrugs, method of inhibiting pathogenic agents in warm-blooded animals, and pharmaceutical compositions comprising deoxyribonucleoside kinase variants of the invention.

### BACKGROUND ART

DNA is made of four deoxyribonucleoside triphosphates, provided by the de novo and the salvage pathway. The key enzyme of the de novo pathway is ribonucleotide reductase, which catalyses the reduction of the 2′-OH group of the nucleoside diphosphates, and the key salvage enzymes are the deoxyribonucleoside kinases, which phosphorylate deoxyribonucleosides to the corresponding deoxyribonucleoside monophosphates.

Deoxyribonucleoside kinases from various organisms differ in their substrate specificity, regulation of gene expression and cellular localisation. In mammalian cells there are four enzymes with overlapping specificities, the thymidine kinases 1 (TK1) and 2 (TK2), deoxycytidine kinase (dCK) and deoxyguanosine kinase (dGK) phosphorylate purine and pyrimidine deoxyribonucleosides. TK1 and TK2 are pyrimidine specific and phosphorylate deoxyuridine (dUrd) and thymidine (dThd), and TK2 also phosphorylates deoxycytidine (dCyd). dCK phosphorylates dCyd, deoxyadenosine (dAdo) and deoxyguanosine (dGuo), but not dThd. dGK phosphorylates dGuo and dAdo. TK1 is cytosolic, and TK2 and dGK are localised in the mitochondria, although recent reports indicate a cytoplasmic localisation of TK2 as well.

In prokaryotic cells, the pattern of deoxyribonucleoside kinases is not very well clarified. In E. coli, there seems to be only one deoxyribonucleoside kinase, which has been characterised as a TK with similarity to the mammalian TK1. The ability to incorporate dCyd, dAdo and dGuo seems to be lacking. In Lactobacillus acidophilus, which is deficient in ribonucleotide reductase, the four deoxyribonucleosides are phosphorylated by three enzymes. In addition to a TK resembling the E. coli TK, there are two kinase complexes that phosphorylate dCyd, dAdo and dGuo. Complex I is a dCK/dAK, and complex II is a dGK/dAK.

Several viruses carry a gene for a TK. Herpes viruses have a TK which also can phosphorylate dCyd as well as TMP and dCMP. The herpetic kinases with the relatively broad substrate specificity have many features in common with the mammalian TK2, dCK and dGK. Poxviruses code for a TK very similar to the mammalian TK1.

So far, however, none of the known viral, bacterial or eukaryotic deoxyribonucleoside kinases were shown to phosphorylate all four deoxyribonucleosides.

Recently a deoxyribonucleoside kinase from Drosophila melanogaster was isolated and named Drosophila melanogaster deoxyribonucleoside kinase, Dm-dNK [Munch-Petersen B, Piskur J, and Søndergaard L: Four Deoxynucleoside kinase Activities from Drosophila melanogaster Are Contained within a Single Monomeric Enzyme, a New Multifunctional Deoxynucleoside Kinase; J. Biol. Chem. 1998 273 (7) 3926-3931]. Subsequently the corresponding gene was cloned and over-expressed [Munch-Petersen B, Knecht W, Lenz C, Søndergaard L and Piskur J: Functional expression of a multi-substrate deoxyribonucleoside kinase from Drosophila melanogaster and its C-terminal deletion mutants; J. Biol. Chem. 2000 275 (9) 6673-6679].

The Drosophila kinase possessed the ability to phosphorylate all four deoxyribonucleosides. This is in sharp contrast to all known deoxyribonucleoside kinases that have distinct, although partially overlapping substrate specificities.

The catalytic rate of deoxyribonucleoside phosphorylation by Dm-dNK was, depending on the substrate, 4-20,000-fold higher than reported for any of the mammalian deoxyribonucleoside kinases. The turnover of thymidine was 70-fold higher than catalysed by the thymidine kinase (TK) of Herpes simplex virus 1 (HSV1). Furthermore, Dm-dNK was able to phosphorylate a wide range of nucleoside analogues used in chemotherapy of cancer or to combat viral infections.

The unique kinetic properties of Dm-dNK make this enzyme interesting for both biotechnological as well as medical applications.

For example, ddNTPs used for sequencing and dNTPs used for PCR—reactions are produced by chemical synthesis with toxic chemicals leading to a number of by-products. Efficient enzymatic synthesis of monophosphates from (di-)deoxyribonucleosides would be one of the key steps in enzymatic production of nucleotides, and Dm-dNK with its broad substrate acceptance and high catalytic rates would be an obvious candidate for this task.

An additional example is the use of deoxyribonucleoside kinases as suicide genes in gene therapy of cancer or in genetic pharmaco-modulation therapy of viral infections. The basic concept here is to transduce cancer or viral infected cells with the gene encoding HSV1-TK and subsequently expose them to a nucleoside analogue. The activation of the nucleoside analogue to a cytotoxic or antiviral compound will be potentiated by the transduced kinase. This concept has demonstrated to increase the effects of cytotoxic or antiviral nucleoside analogues in combination with HSV1-TK, human deoxycytidine kinase (dCK) and human deoxyguanosine kinase (dGK). The key step in activation of the majority of the nucleoside analogues is the conversion to the monophosphate.

Therefore the kinetic properties of the enzymes catalysing this step are important both for the efficacy and selectivity of these drugs and there is a need to identify better enzymes for further development of this therapeutic concept. Dm-dNK with its unique kinetic properties has been proposed as a candidate for this purpose [Johansson M, Van Rompay A R, Degreves B, Balzarini J and Karlsson A: Cloning and characterization of the multisubstrate deoxynucleoside kinase of Drosophila melanogaster, J. Biol. Chem. 1999 274 (34) 23814-23819; and Munch-Petersen et al.; J. Biol. Chem. 2000 275 (9) 6673-6679].

Recently, in an effort to find better suicide gene-prodrug combinations for gene therapy, mutants of HSV1-TK with improved specificity for the nucleoside analogues 3′-azido-2′,3′-dideoxythymidine (Zidovudine, Retrovir®, AZT), ganciclovir (Cytovene®, GCV) and aciclovir (Zovirax®, ACV) have been genetically engineered by primer mediated random mutagenesis or DNA family shuffling [Black M E, Newcomb T G, Wilson H M P and Loeb L A: Creation of drug-specific herpes simplex virus type 1 thymidine kinase mutants for gene therapy; Proc. Natl. Acad. Sci. USA 1996 93 3523529; Christians F C, Scapozza L, Crameri A, Folkers G and Stemmer W P C: Directed evolution of thymidine kinase for AZT phosphorylation using DNA family shuffling; Nat. Biotechnol. 1999 17 259-264; and Kokons M S, Sabo P, Adman E T and Black M E: Enhancement of tumor ablation by a selected HSV-1 thymidine kinase mutant; Gene Therapy 1999 6 1415-1426].

Nucleoside analogues with changes in the 2′-deoxyribose moiety are important drugs in medicine and precursors for nucleotides frequently used in biotechnology.

### SUMMARY OF THE INVENTION

It is an object of the present invention to provide novel deoxyribonucleoside kinase variants with increased relative catalytic efficiencies towards different substrates. This object is met by the provision of novel multi-substrate deoxyribonucleoside kinase variants.

Accordingly, in its first aspect, the invention provides isolated, mutated polynucleotides encoding multi-substrate deoxyribonucleoside kinase enzymes, which mutated polynucleotide, when compared to the non-mutated polynucleotide, and upon transformation into a bacterial or eukaryotic cell, decreases at least 4 fold the lethal dose (LD₁₀₀) of at least one nucleoside analogue.

In another aspect the invention provides isolated deoxyribonucleoside kinase variants encoded by the polynucleotide of the invention.

In a third aspect the invention provides vector constructs comprising the polynucleotide of the invention.

In a fourth aspect the invention provides packaging cell lines capable of producing an infective virion comprising comprising a viral vector of the invention.

In a fifth aspect the invention provides host cells carrying the mutated polynucleotide of the invention, or the vector of the invention.

In a sixth aspect the invention provides methods of sensitising cells to prodrugs, which methods comprises the steps of transfecting said cell with a polynucleotide sequence of the invention encoding an enzyme that promotes the conversion of said prodrug into a (cytotoxic) drug; and delivering said prodrug to said cell; wherein said cell is more sensitive to said (cytotoxic) drug than to said prodrug.

In a seventh aspect the invention provides methods of inhibiting pathogenic agents in warm-blooded animals, which methods comprises administering to said animals a mutated polynucleotide of the invention, or a vector of the invention.

In an eight aspect the invention provides pharmaceutical compositions comprising a mutated polynucleotide of the invention, or a vector of the invention.

In a nineth aspect the invention provides pharmaceutical compositions comprising the enzyme variant of the invention, and a pharmaceutically acceptable carrier or diluent.

Other objects of the invention will be apparent to the person skilled in the art from the following detailed description and examples.

### DETAILED DISCLOSURE OF THE INVENTION

Mutant Polynucleotides

In its first aspect the invention provides isolated, mutated polynucleotides encoding insect or lower vertebrate deoxyribonucleoside kinase enzymes.

The mutant polynucleotides of the invention include DNA, cDNA and RNA sequences, as well as anti-sense sequences, and include naturally occurring, synthetic, and intentionally manipulated polynucleotides. The mutant polynucleotides of the invention also include sequences that are degenerate as a result of the genetic code.

As defined herein, the term “polynucleotide” refers to a polymeric form of nucleotides of at least 10 bases in length, preferably at least 15 bases in length. By “isolated polynucleotide” is meant a polynucleotide that is not immediately contiguous with both of the coding sequences with which it is immediately contiguous (one on the 5′ end and one on the 3′ end) in the naturally occurring genome of the organism from which it is derived. The term therefore includes recombinant DNA which is incorporated into an expression vector, into an autonomously replicating plasmid or virus, or into the genomic DNA of a prokaryote or eukaryote, or which exists as a separate molecule, e.g. a cDNA, independent from other sequences.

As defined herein a mutant polynucleotide is a nucleotide sequence that differs at one or more nucleotide positions when compared to the non-mutated (native, wild-type or parent) nucleotide sequence. The mutated polynucleotide of the invention may in particular hold a nucleotide sequence encoding a nucleoside kinase variant having an amino acid sequence that has been changed at one or more positions when compared to the native, wild-type or parent kinase enzyme.

In a preferred embodiment the mutated polynucleotide holds a nucleotide sequence encoding a nucleoside kinase variant having an amino acid sequence that has been changed at one or more positions located in the non-motif regions, and/or at only one motif region, as defined by Table 1, below.

In another preferred embodiment the mutated polynucleotide of the invention, upon transformation into a bacterial or eukaryotic cell, is capable of decreasing at least 4 fold, more preferred at least 8 fold, most preferred at least 10 fold the lethal dose (LD₁₀₀) of at least one nucleoside analogue, as compared to the non-mutated (wild-type) polynucleotide. In a more preferred embodiment the nucleoside analogue is aciclovir (9-[2-hydroxy-ethoxy]-methyl-guanosine), buciclovir, famciclovir, ganciclovir (9-[2-hydroxy-1-(hydroxymethyl)ethoxyl-methyl]-guanosine), penciclovir, valciclovir, trifluorothymidine, AZT (3′-azido-3′-deoxythymidine), AIU (5′-iodo-5′-amino-2′,5′-dideoxyuridine), ara-A (adenosine-arabinoside; Vivarabine), ara-C (cytidine-arabinoside), ara-G (9-beta-D-arabinofuranosylguanine), ara-T, 1-beta-D-arabinofuranosyl thymine, 5-ethyl-2′-deoxyuridine, 5-iodo-5′-amino-2,5′-dideoxyuridine, 1-[2-deoxy-2-fluoro-beta-D-arabino furanosyl]-5-iodouracil, idoxuridine (5-iodo-2′deoxyuridine), fludarabine (2-Fluoroadenine 9-beta-D-Arabinofuranoside), gencitabine, 2′,3′-dideoxyinosine (ddI), 2′,3′-dideoxycytidine (ddC), 2′,3′-dideoxythymidine (ddT), 2′,3′-dideoxyadenosine (ddA), 2′,3′-dideoxyguanosine (ddG), 2-chloro-2′-deoxyadenosine (2CdA), 5-fluorodeoxyuridine, BVaraU ((E)-5-(2-bromovinyl)-1-beta-D-arabinofuranosyluracil), BVDU (5-bromovinyl-deoxyuridine), FIAU (1-(2-deoxy-2-fluoro-beta-D-arabinofuranosyl)-5-iodouracil), 3TC (2′-deoxy-3′-thiacytidine), dFdC gemcitabine -(2′,2′-difluorodeoxycytidine), dFdG (2′,2′-difluorodeoxyguanosine), or d4T (2′,3′didehydro-3′-deoxythymidine).

In yet another preferred embodiment the mutated polynucleotide of the invention, upon transformation into a bacterial or eukaryotic cell, is capable of decreasing at least 4 fold, preferably at least 8 fold, most preferred at least 10 fold, the lethal dose (LD₁₀₀) of at least two different nucleoside analogues, which analogous are based on two different sugar moieties and two different base moieties.

In a preferred embodiment, the mutated polynucleotide of the invention has the DNA sequence presented as SEQ ID NOS: 9 or 11.

Enzyme Variants

In another aspect the invention provides substantially pure deoxyribonucleoside kinase variants.

In the context of this invention, the term “enzyme variant” covers a polypeptide (or a protein) having an amino acid sequence that differs from that of the native, parent or wild-type enzyme at one or more amino acid positions, i.e. its primary amino acid sequence has been modified. Such enzyme variants include the variants described in more detail below, as well as conservative substitutions, splice variants, isoforms, homologues from other species, and polymorphisms.

The novel enzyme variants of the invention may in particular be obtained from a mutated polynucleotide of the invention using standard recombinant DNA technology.

In a preferred embodiment enzyme variants of the invention invention are derived from a multi-substrate kinase. As defined herein, the term “multi-substrate” refers to a deoxyribonucleoside kinase enzyme capable of having the ability to phosphorylate all four native nucleosides, dC, dA, dG and dT (Thd). The ability to phosphorylate all four native nucleosides may be determined by the ratio of maximal specific enzyme activity (enzyme activity/amount of enzyme) for dT, and for any of these nucleosides (maximal specific enzyme activity for dT/maximal specific enzyme activity for dC, dG or dA). This ratio preferably is in the range of from 0.01 to 100.

In a preferred embodiment the enzyme variant of the invention, in comparison to the wild-type enzyme, has been altered with respect to

(i) the ratio “k_{cat}/Kₘ(substrate)/k_{cat}/Kₘ(nucleoside analogue)” (i.e. the ratio between on the one side “k_{cat}/Kₘ” for at least one native substrate, and on the other side “k_{cat}/Kₘ” for at least one nucleoside analogue) is decreased by at least at least 5 fold, more preferred at least 10 fold, most preferred at least 20 fold; and/or

(ii) the feedback inhibition by deoxyribonucleoside triphosphate (dNTP), and in particular thymidine triphosphate (TTP), is decreased by at least 1.5 fold, more preferred at least 2 fold, as determined by its IC₅₀ value using 2 or 10 μM thymidine (dThd) as a substrate.

In a preferred embodiment the enzyme variant of the invention, in comparison to the wild-type enzyme, decreases at least 4 fold, preferably at least 8 fold, most preferred at least 10 fold, the lethal dose (LD₁₀₀) of at least two different nucleoside analogues, which analogous are based on two different sugar moieties and two different base moieties.

dNK Numbering System

In the context of this invention, amino acid residues (as well as nucleic acid bases) are specified using the established one-letter symbol.

By aligning the amino acid sequences of the known deoxyribonucleoside kinase enzymes, a specific amino acid numbering system may be employed, by which system it is possible to unambiguously allot an amino acid position number to any amino acid residue in any nucleoside kinase enzyme, which amino acid sequence is known.

Such an alignment is presented in Table 1, below. In this table, the first N-terminal amino acid residue (i.e. methionine; M) of Dm-dNK carries number 51, and the last C-terminal amino acid residue (i.e. arginine; R) of Dm-dNK carries number 358.

In the context of this invention this numbering system is designated the dNK Numbering System.

In describing the various enzyme variants produced or contemplated according to the invention, the following nomenclatures have been adapted for ease of reference:

### Original Amino Acid/Position/Substituted Amino Acid

According to this nomenclature the substitution of alanine for valine at position 167 is designated as “V167A”.

A deletion of methionine at position 51 is designated “M51*”.

An insertion of an additional amino acid residue, in this example arginine, e.g. adjacent to position 62, may be designated “T62TR” or “*63R” (assumed that no position exists for this position in the amino acid sequence used for establishing the numbering system).

An insertion of an amino acid residue, in this example glutamine, at a position which exists in the established numbering system, but where no amino acid residue is actually present, may be designated “-116Q”.

In this way “Dm-dNK/I199M/N216S/M217V/D316N” specifies the particular variant that may be derived from the Drosophila melanogaster deoxyribonucleoside kinase by substitution of methionine for isoleucine at position 199, and substitution of serine for asparagine at position 216, and substitution of valine for methionine at position 217, and substitution of asparagine for aspartic acid at position 316, the positions being determined in accordance with Table 1 below.

Other enzyme variants, derived from the same or from different sources, are identified in the same manner.

TABLE 1
Multiple Sequence Alignment
dNK Numbering

| | | |
|---|---|---|
| Dm-dNK | ---------- ---------- ---------- ---------- ---------- MAEAASCARK | 060 |
| BmK | ---------- ---------- ---------- ---------- ---------- ---------- | |
| XenK | MSVLLAARTC IRLCCTEHKT GALARFNLGA NTALTVRRIA SALCG-RCNI MRRGILPSGS | |
| hu-TK2 | ---------- ---------- ---------- ---------- ---------- --MGAFCQRP | |
| hu-dGK | ---------- ---------- ---------- --MAAGRLFL SRLRA-PFSS MAKSPLEGVS | |
| hu-dCK | ---------- ---------- ---------- ---------- ---------- MATPPKRSCP | |
| HSV1-TK | ---------- ---------- ----MASYPG HQHASAFDQA ARSRGHSNRR TALRPRRQQE | |
| Dm-dNK | GT-KYAEGTQ P--FTVLIEG NIGSGKTTYL NHFEKY--KN DICLLTEPVE KWRNV----- | 120 |
| BmK | ---MSANNVK P--FTVFVEG NIGSGKTTFL EHFRQF--E- DITLLTEPVE MWRDL----- | |
| XenK | TGNGLKSREK S--TVICVEG NIASGKTSCL DYFSNT--P- DLEVFKEPVA KWRNV----- | |
| hu-TK2 | SSDKEQEKEK K--SVICVEG NIAGGKTTCL EFFSNA--T- DVEVLTEPVS KWRNV----- | |
| hu-dGK | SSRGLHAGRG P--RRLSIEG NIAVGKSTFV KLLTKT--YP EWHVATEPVA TWQNIQAAGN | |
| hu-dCK | SFSASSEGTR I--KKISIEG NIAAGKSTFV NILKQL--CE DWEVVPEPVA RWCNVQSTQD | |
| HSV1-TK | ATEVRPEQKM PTLLRVYIDG PHGMGKTTTT QLLVALGSRD DIVYVPEPMT YWRVLGAS-- | |
| | . ..* **.. . **. * . | |
| | Motif 1 Motif 2 | |
| Dm-dNK | ---------- NGVNLLELMY K-DP------ ------KKWA MPFQSYVTLT M--LQSHTAP | 180 |
| Bm-dNK | ---------- KGCNLLELMY K-DP------ ------EKWA MTFQSYVSLT M--LDMHRRP | |
| Xen-dNK | ---------- CGHNPLGLMY Q-DP------ ------NKWG LTLQTYVQLT M--LDIHTKP | |
| hu-TK2 | ---------- RGHNPLGLMY H-DA------ ------SRWG LTLQTYVQLT M--LDRHTRP | |
| hu-dGK | ---QKACTAQ SLGNLLDMMY R-EP------ ------ARWS YTFQTFSFLS R--LKVQLEP | |
| hu-dCK | EFEELTMSQK NGGNVLQMMY E-KP------ ------ERWS FTFQTYACLS R--IRAQLAS | |
| HSV1-TK | ---------- ---ETIANIY TTQHRLDQGE ISAGDAAVVM TSAQITMGMP YAVTDAVLAP | |
| | . . .* . * . | |
| Dm-dNK | TNKKLK---- --------IM ERSIFSAR-- YCFVENMRRN GSLEQGMYNT LEEWYKFIEE | 240 |
| Bm-dNK | APTPVK---- --------LM ERSLFSAR-- YCFVEHIMRN NTLHPAQFAV LDEWFRFIQH | |
| Xen-dNK | SISPVK---- --------MM ERSIYSAK-- YIFVENLYQS GKMPAVDYAI LTEWFKWIVK | |
| hu-TK2 | QVSSVR---- --------LM ERSIHSAR-- YIFVENLYRS GKMPEVDYVV LSEWFDWILR | |
| hu-dGK | FPEKLLQ--- ARKPVQ--IF ERSVYSDR-- YIFAKNLFEN GSLSDIEWHI YQDWHSFLLW | |
| hu-dCK | LNGKLKD--- AEKPVL--FF ERSVYSDR-- YIFASNLYES ECMNETEWTI YQDWHDWMNN | |
| HSV1-TK | HIGGEAGSSH APPPALTLIF DRHPIAALLC-YPAARYLMGS MTPQAVLAFV ALIPPTLFGT | |
| | .. .* . * . | |
| | Motif 3 Motif 4 | |
| Dm-dNK | SIHVQADL-- IIYLRTSPEV AY-ERIRQRA RSEESCVPLK YLQELHELHE DWLIHQRR-- | 300 |
| Bm-dNK | NIPIDADL-- IVYLKTSPSI VY-QRIKKRA RSEEQCVPLS YIEELHRLHE DWLINRIH-- | |
| Xen-dNK | NTDTSVDL-- IVYLQTSPEI CY-QRLKKRC REEESVIPLE YLCAIHNLYE DWLVKQTS-- | |
| hu-TK2 | NMDVSVDL-- IVYLRTNPET CY-QRLKKRC REEEKVIPLE YLEAIHHLHE EWLIKGSL-- | |
| hu-dGK | EFASRITLHG FIYLQASPQV CL-KRLYQRA REEEKGIELA YLEQLHGQHE AWLIHKTTKL | |
| hu-dCK | QFGQSLELDG IIYLQATPET CL-HRIYLEG RNEEQGIPLE YLEKLHYKHE SWLLHRTLKT | |
| HSV1-TK | NIVLGAL--- -------PED RHIDRLAKRQ RPGER-LDLA MLAAIRRVYG --LLANTVRY | |
| | * *. * * * . * * .. . *. | |
| | Motif 5 | |
| Dm-dNK | ----PQSCKV LVLDADLNLE NIGTEYQRSE SSIFDAISSN QQPSPVLVSP SKRQRVAR-- | 360 |
| Bm-dNK | ---AECPAPV LVLDADLDLS QITDEYKRSE HQILRKAVNV VMSSPNKHSP KKPISTTPIK | |
| Xen-dNK | ---FSVPAPV LVIDGNKELE ELTQHYEENR TSILSL---- ---------- ---------- | |
| hu-TK2 | ---FPMAAPV LVIEADHHME RMLELFEQNR DRILTPENRK HCP------- ---------- | |
| hu-dGK | HFEALMNIPV LVLDVNDDFS EE-VTKQEDL MREVNTFVKNL --------- ---------- | |
| hu-dCK | NFDYLQEVPI LTLDVNEDFK D----KYESL VEKVKEFLSTL --------- ---------- | |
| HSV1-TK | LQCGGSWRED WGQLSGTAVP PQGAEPQSNA GPRPHIGDTLF TLFRAPEL LAPNGDLYNV | |
| Dm-dNK | -------- 370 | |
| Bm-dNK | ITPHMRIL | |
| Xen-dNK | -------- | |
| hu-TK2 | -------- | |
| hu-dGK | -------- | |
| hu-dCK | -------- | |
| HSV1-TK | FAWALDVL ( . . . continued) | |

Dm-dNK Drosophila melanogaster deoxyribonucleoside kinase [Munch-Petersen B, Knecht W, Lenz C, Sndergaard L and Piskur J; J. Biol. Chem. 2000 275 (9) 6673-6679; GenBank ACCN AF226281; Presented as SEQ ID NO: 1]
Bm-dNK Bombyx mori deoxyribonucleoside kinase [GenBank ACCN AF226281; Presented as SEQ ID NO: 3, obtained as described in Example 3]
Xen-dNK Xenopus laevis deoxyribonucleoside kinase [GenBank ACCN AF250861; Presented as SEQ ID NO: 5, obtained as described in Example 3]
hu-TK2 Human thymidine kinase 2 [GenBank ACCN O00142; Johansson M & Karlsson A; J. Biol. Chem. 1997 272 (13) 8454-8458]
hu-dGK Human deoxyguanosine kinase [GenBank ACCN Q19854; Johansson M & Karlsson A; Proc. Natl. Acad. Sci. U.S.A. 1996 93 (14) 7258-7262]
hu-dCK Human deoxycytidine kinase [GenBank ACCN P27707; Chottiner, E. G., et al.; Proc. Natl. Acad. Sci. U.S.A. 1991 88 (4) 1531-1535]
HSV1-TK Herpes simplex virus thymidine kinase [GenBank ACCN CAA23742; McKnight SL; Nucleic Acids Res. 1980 8 (24) 5949-5964]
“Motif” designates a preserved motif of amino acids
- indicates absent (no) amino acid at this position.
* indicates positions which have a single, fully conserved residue.
. indicates that one of the following “conservative” groups is fully conserved: -STA, NEQK, NHQK, NDEQ, QHRK, MILV, MILF, HY or FYW.

In another preferred embodiment, the enzyme variant of the invention, when compared to the wild-type enzyme, has been mutated

(i) in a non-motif and/or a non-conserved region; and/or

(ii) in only one motif and/or conserved region; and/or

(iii) in any conserved position.

In a yet more preferred embodiment, the enzyme variant of the invention, when compared to the wild-type enzyme, has been mutated

(i) in a non-motif; and/or

(ii) in only one motif region; and/or

(iii) in any conserved position.

As defined herein a motif region designates any of the positions located within the any of the five motif regions identified in Table 1 above. A non-motif region is any region containing amino acid residues not belonging to a motif region as defined above.

As defined herein conserved positions are those positions and regions containing the amino acid residues marked with an asterisk (*) or with a period (.) in Table 1. In a preferred embodiment the conserved region is selected from those regions containing amino acid residues marked with an asterisk (*) only, i.e. those holding a single fully conserved residue. A non-conserved region is any region containing amino acid residues not belonging to the conserved positions as defined above.

In another preferred embodiment, the enzyme variant of the invention, when compared to the wild-type enzyme, holds a mutation (incl. substitutions, additions and deletions) at one or more of the following positions 51, 62, 82, 91, 100, 102, 107, 112, 114, 134, 138, 139, 140, 164, 167, 168, 171, 199, 202, 207, 211, 213, 214, 216, 217, 220, 222, 228, 229, 274, 277, 281, 283, 284, 307, 309, 316, 318, 321, 334, 347, and 352 (dNK numbering).

In a more preferred embodiment the enzyme variant of the invention, when compared to the wild-type enzyme, comprises a substitution conservative to those of G80, N81, I82, G83, S84, G85, K86, T87, T88, E107, P108, V109, E110, K111, W112, Y140, Q164, E201, R202, S203, C210, Y211, C212, P258, R265, I266, R267, Q268, R269, A270, R271, E274, L279, I282, or L293 (dNK numbering).

As defined herein, the term “conservative substitutions” denotes the replacement of an amino acid residue by another, biologically similar residue. Examples of conservative substitutions include

(i) the substitution of one non-polar or hydrophobic residue such as alanine, leucine, isoleucine, valine, proline, methionine, phenylalanine or tryptophan for each other, in particular the substitution of alanine, leucine, isoleucine, valine or proline for each other; or

(ii) the substitution of one neutral (uncharged) polar residue such as serine, threonine, tyrosine, asparagine, glutamine, or cysteine for another, in particular the substitution of arginine for lysine, glutamic for aspartic acid, or glutamine for asparagine; or

(iii) the substitution of a positively charged residue such as lysine, arginine or histidine for another; or

(iv) the substitution of a negatively charged residue such as aspartic acid or glutamic acid for another.

The term conservative substitution also includes the use of a substituted amino acid residue in place of a parent amino acid residue, provided that antibodies raised to the substituted polypeptide also immuno-react with the un-substituted polypeptide.

In a yet more preferred embodiment the enzyme variant of the invention, when compared to the wild-type enzyme, comprises one or more of the following variations M51T; T62A; N91D; N100D; I102T; N114D; N134D; N134S; L138S; M139L; M139V; V167A; V167S; V167M; T168A; M171R; I199M A207D; V214A; N216S; M217V; N220S; S222W; Y228C; N229S; V277A; Y281H; S307P; K309R; D316N; N318D; N321S; F334L; L347P; and K352N (dNK numbering).

In an even more preferred embodiment the enzyme variant of the invention, when compared to the wild-type enzyme, comprises the following variations

M51T/T168A/N220S;

T62A/V167A/N321S;

N91D/N134D;

N100D/N134D;

N100D/N134D/N318D/L347P;

N100D/N134D/I199M/N216S/M217V/D316N;

I102T/N318D;

N114D/M217V/Y281H;

N134S/L138S/M139L/K352N;

M139V/N318D/L347P;

V167A/M171R/A207D;

V167S/M171R/A207D;

V167A/I199M/N216S/M217V/D316N;

V167A/N318D/L347P;

T168A/N318D/L347P;

T168A/I199M/N216S/M217V/D316N;

M171R/A207D;

I199M/V214A/N216S/M217V/D316N;

I199M/N216S/M217V/N229S/S307P/D316N;

I199M/N216S/M217V/D316N;

S222W/F334L;

Y228C/V277A/K309R; or

N318D/L347P (dNK numbering).

In a preferred embodiment the enzyme variant of the invention is derived from a human thymidine kinase 2 (hu-TK2); or a human deoxyguanosine kinase (hu-dGK); or a human deoxycytidine kinase (hu-dCK); or a Herpes simplex virus thymidine kinase (HSV1-TK).

In another preferred embodiment the enzyme variant of the invention is derived from an insect or a lower vertebrate, in particular from a Drosophila melanogaster deoxyribonucleoside kinase (Dm-dNK), or a Bombyx mori deoxyribonucleoside kinase (Bm-dNK), or a Xenopus laevis deoxyribonucleoside kinase (Xen-dNK), or an Anopheles gambia deoxyribonucleoside kinase.

In a more preferred embodiment the enzyme variant of the invention is Dm-dNK/M51T; Dm-dNK/M51T/T168A/N220S; Dm-dNK/T62A; Dm-dNK/T62A/V167A/N321S; Dm-dNK/N91D; Dm-dNK/N91D/N134D; Dm-dNK/N100D; Dm-dNK/N100D/N134D; Dm-dNK/N100D/N134D/N318D/L347P; Dm-dNK/N100D/N134D/I199M/N216S/M217V/D316N; Dm-dNK/I102T; Dm-dNK/I102T/N318D; Dm-dNK/N114D; Dm-dNK/N114D/M217V/Y281H; Dm-dNK/N134D; Dm-dNK/N134S; Dm-dNK/N134S/L138S/M139L/K352N; Dm-dNK/L138S; Dm-dNK/M139L; Dm-dNK/M139V; Dm-dNK/M139V/N318D/L347P; Dm-dNK/V167A; Dm-dNK/V167A/I199M/N216S/M217V/D316N; Dm-dNK/V167A/N318D/L347P; Dm-dNK/T168A; Dm-dNK/V167A/M171R/A207D, Dm-dNK/V167S/M171R/A207D, Dm-dNK/T168A/N318D/L347P; Dm-dNK/T168A/I199M/N216S/M217V/D316N; Dm-dNK/M171R/A207D Dm-dNK/I199M; Dm-dNK/I199M/V214A/N216S/M217V/D316N; Dm-dNK/I199M/N216S/M217V/N229S/S307P/D316N; Dm-dNK/I199M/N216S/M217V/D316N; Dm-dNK/V214A; Dm-dNK/N216S; Dm-dNK/M217V; Dm-dNK/N220S; Dm-dNK/S222W; Dm-dNK/S222W/F334L; Dm-dNK/Y228C; Dm-dNK/Y228C/V277A/K309R; Dm-dNK/N229S; Dm-dNK/V277A; Dm-dNK/Y281H; Dm-dNK/S307P; Dm-dNK/K309R; Dm-dNK/D316N; Dm-dNK/N318D; Dm-dNK/N318D/L347P; Dm-dNK/N321S; Dm-dNK/F334L; Dm-dNK/L347P; or Dm-dNK/K352N (dNK-numbering).

In another preferred embodiment the enzyme variant of the invention is; Bm-dNK/E91D; Bm-dNK/E91D/N134D; Bm-dNK/-100D; Bm-dNK/-100D/N134D; Bm-dNK/-100D/N134D/K347P; Bm-dNK/-100D/N134D/L199M/H216S/I217V/D316N; Bm-dNK/I102T; Bm-dNK/N114D; Bm-dNK/N114D/I217V/Y281H; Bm-dNK/N134D; Bm-dNK/N134S; Bm-dNK/N134S/L138S/M139L/K352N; Bm-dNK/L138S; Bm-dNK/M139L; Bm-dNK/M139V; Bm-dNK/M139V/K347P; Bm-dNK/V167A; Bm-dNK/V167A/L199M/H216S/I217V/D316N; Bm-dNK/V167A/Q321S; Bm-dNK/V167A/K347P; Bm-dNK/V167A/M171R/A207D, Bm-dNK/V167S/M171R/A207D, Bm-dNK/S168A; Bm-dNK/S168A/L199M/H216S/I217V/D316N; Bm-dNK/S168A/N220S; Bm-dNK/S168A/K347P; Bm-dNK/M171R/A207D; Bm-dNK/L199M; Bm-dNK/L199M/H216S/I217V/D316N; Bm-dNK/L199M/V214A/H216S/I217V/D316N; Bm-dNK/I199M/H216S/I217V/A229S/D316N; Bm-dNK/V214A; Bm-dNK/H216S; Bm-dNK/I217V; Bm-dNK/N220S; Bm-dNK/T222W; Bm-dNK/F228C; Bm-dNK/F228C/V277A/P309R; Bm-dNK/V277A; Bm-dNK/A229S; Bm-dNK/Y281H; Bm-dNK/P309R; Bm-dNK/D316N; Bm-dNK/Q321S; Bm-dNK/L334L; Bm-dNK/K347P; or Bm-dNK/K352N (dNK numbering).

In a third preferred embodiment the enzyme variant of the invention is Xen-dNK/M51T; Xen-dNK/M51T/Q168A; Xen-dNK/G62A; Xen-dNK/G62A/V167A/E321S; Xen-dNK/-100D; Xen-dNK/-100D/N134D; Xen-dNK/-100D/N134D/E318D; Xen-dNK/-100D/N134D/N216S/L217V; Xen-dNK/L102T; Xen-dNK/L102T/E318D; Xen-dNK/N114D; Xen-dNK/N114D/L217V/Y281H; Xen-dNK/N134D; Xen-dNK/N134S; Xen-dNK/N134S/L138S/M139L; Xen-dNK/L138S; Xen-dNK/M139L; Xen-dNK/M139V; Xen-dNK/M139V/E318D/; Xen-dNK/V167A; Xen-dNK/V167A/N216S/L217V; Xen-dNK/V167A/E318D; Xen-dNK/V167A/M171R/A207D, Xen-dNK/V167S/M171R/A207D, Xen-dNK/Q168A; Xen-dNK/Q168A/N216S/L217V; Xen-dNK/Q168A/E318D; Xen-dNK/M171R/A207D; Xen-dNK/V214A; Xen-dNK/V214A/N216S/L117V; Xen-dNK/N216S; Xen-dNK/N216S/L217V; Xen-dNK/N216S/L217N/A229S; Xen-dNK/L217V; Xen-dNK/K222W; Xen-dNK/Y228C; Xen-dNK/Y228C/I277A/P309R; Xen-dNK/A229S; Xen-dNK/I277A; Xen-dNK/Y281H; Xen-dNK/P309R; Xen-dNK/E318D; or Xen-dNK/E321S (dNK numbering).

Hybrid Enzymes

In a particularly preferred embodiment, the deoxyribonucleoside kinase variant of the invention may be a hybrid deoxyribonucleoside kinase derived from two or more insect multi-substrate deoxyribonucleoside kinases.

The hybrid deoxyribonucleoside kinase of the inventionshould contain at least 5, preferably at least 10, more preferred at least 15, even more preferred at least 20, most preferred at least 25 consecutive amino acids derived from each insect multi-substrate deoxyribonucleoside kinases.

In a preferred embodiment the hybrid kinase enzyme is derived from a Drosophila melanogaster deoxyribonucleoside kinase, and/or a Bombyx mori deoxyribonucleoside kinase, and/or a Xenopus laevis deoxyribonucleoside kinase, and/or an Anopheles gambia deoxyribonucleoside kinase.

In a more preferred embodiment, the hybrid kinase enzyme of the invention is derived from a Drosophila melanogaster deoxyribonucleoside kinase and a Bombyx mori deoxyribonucleoside kinase, and comprises the amino acid sequence presented as SEQ ID NO: 10, or the amino acid sequence presented as SEQ ID NO: 12.

Recombinant Vectors

Within another aspect the invention provides a recombinant vector comprising the mutant polynucleotide of the invention.

As defined herein, a recombinant vector is an expression vehicle or recombinant expression construct used for introducing polynucleotides into a desired cell. The expression vector may be a virus vector or a plasmid vector, in which the polynucleotide of the invention may be inserted in a forward or reverse orientation. The vector may also be a synthetic gene.

Suitable expression vehicles include, but are not limited to eukaryotic vectors, prokaryotic vectors, e.g. bacterial linear or circular plasmids, viral vectors, DNA-protein complexes, e.g. DNA-monoclonal antibody complexes, and receptor-mediated vectors. The vector may in particular be contained within a liposome.

Preferred bacterial vectors include pQE30, pQE70, pQE60, pQE-9 (available from Quigen); pbs, pD10, phagescript, psiX174, pbluescript SK, pbsks, pNH8A, pNH16A, pNH18A, pNH46A (available from Stratagene); pGEX-2T, PKK223-3, pKK233-3, pDR540 and pRIT5 (available from Pharmacia); and pASK75 (available from Biometra).

Preferred eukaryotic vectors include pWLNEO, pSV2CAT, pOG44, pXT1, pSG (available from Stratagene); pSVK3, pBPV, pMSG, pSVL (available from Pharmacia); and pTEJ-8 [FEBS Lett. 1990 267 289-294] and pcDNA-3 (available from Invitrogen). Preferred yeast vectors include pYES2 (available from Invitrogen).

Preferred viral vectors include herpes simplex viral vectors, adenoviral vectors, adenovirus-associated viral vectors, pox vectors, parvoviral vectors, baculovirus vectors and retroviral vectors.

However, any other plasmid or vector may be used as long as they are replicable and viable in the production host.

The expression vector may further comprise regulatory sequences in operable combination with the polynucleotide sequence of the invention. As defined herein, the term “in operable combination” means that the operable elements, i.e. gene(s) and the regulatory sequences, are operably linked so as to effect the desired expression. Promoters are examples of such regulatory sequences.

In a preferred embodiment the vector of the invention comprises a promoter operably linked to the polynucleotide.

The regulatory elements may be selected from any desired source and the vector produced using standard techniques known in the art, e.g. those described by Sambrook et al. [Sambrook et al.: Molecular Cloning: A Laboratory Manual, Cold Spring Harbor Lab., Cold Spring Harbor, N.Y. 1989].

In a preferred embodiment, the vector is a viral vector, in particular a herpes simplex viral vector, an adenoviral vector, an adenovirus-associated viral vector, or a retroviral vector. The choice of vector and its regulatory elements of course depends on the purpose of the expression, and is within the discretion of the person skilled in the art.

In yet another aspect the invention provides packaging cell lines capable of producing an infective virion comprising the virus vector of the invention.

Host/Production Cells

In a yet further aspect the invention provides a production cell genetically manipulated to comprise the polynucleotide sequence of the invention, and/or or a recombinant expression vector of the invention. The cell of the invention may in particular be genetically manipulated to transiently or stably express, over-express or co-express polypeptide of the invention. Methods for generating transient and stable expression are known in the art.

The polynucleotide of the invention may be inserted into an expression vector, e.g. a plasmid, virus or other expression vehicle, and operatively linked to expression control sequences by ligation in a way that expression of the coding sequence is achieved under conditions compatible with the expression control sequences. Suitable expression control sequences include promoters, enhancers, transcription terminators, start codons, splicing signals for introns, and stop codons, all maintained in the correct reading frame of the polynucleotide of the invention so as to permit proper translation of mRNA. Expression control sequences may also include additional components such as leader sequences and fusion partner sequences.

The promoter may in particular be a constitutive or an inducible promoter. When cloning in bacterial systems, inducible promoters such as pL of bacteriophage λ, plac, ptrp, ptac (ptrp-lac hybrid promoter), may be used. When cloning in mammalian systems, promoters derived from the genome of mammalian cells, e.g. the ubiquitin promoter, the TK promoter, or the metallothionein promoter, or from mammalian viruses, e.g. the retrovirus long terminal repeat, the adenovirus late promoter or the vaccinia virus 7.5K promoter, may be used. Promoters obtained by recombinant DNA or synthetic techniques may also be used to provide for transcription of the polynucleotide of the invention.

Suitable expression vectors typically comprise an origin of expression, a promoter as well as specific genes which allow for phenotypic selection of the transformed cells, and include vectors like the T7-based expression vector for expression in bacteria [Rosenberg et at, Gene 1987 56 125], the pTEJ-8, pUbi1Z, pcDNA-3 and pMSXND expression vectors for expression in mammalian cells [Lee and Nathans, J. Biol. Chem. 1988 263 3521], baculovirus derived vectors for expression in insect cells, and the oocyte expression vector PTLN [Lorenz C, Pusch M & Jentsch T J: Heteromultimeric CLC chloride channels with novel properties; Proc. Natl. Acad. Sci. USA 1996 93 13362-13366].

In a preferred embodiment, the cell of the invention is an eukaryotic cell, e.g., a mammalian cell, e.g., a human cell, a dog cell, a monkey cell, a rat cell or a mouse cell, an oocyte, or a yeast cell. The cell of the invention may be without limitation a human embryonic kidney (HEK) cell, e.g., a HEK 293 cell, a BHK21 cell, a Chinese hamster ovary (CHO) cell, a Xenopus laevis oocyte (XLO) cell. In another embodiment, the cell of the invention is a fungal cell, e.g., a filamentous fungal cell. In another preferred embodiment, the cell is an insect cell, most preferably the Sf9 cell. Additional preferred mammalian cells of the invention are PC12, HiB5, RN33b cell lines and human neural progenitor cells. Most preferred are human cells.

When the cell of the invention is an eukaryotic cell, incorporation of the heterologous polynucleotide of the invention may in particular be carried out by infection (employing a virus vector), by transfection (employing a plasmid vector), using calcium phosphate precipitation, microinjection, electroporation, lipofection, or other physical-chemical methods known in the art.

In a more preferred embodiment the isolated polynucleotide sequence of the invention, and/or or a recombinant expression vector of the invention are transfected in a mammalian host cell, a neural progenitor cell, an astrocyte cell, a T-cell, a hematopoitic stem cell, a non-dividing cell, or a cerebral endothelial cell, comprising at least one DNA molecule capable of mediating cellular immortalization and/or transformation.

Activation of an endogenous gene in a host cell may be accomplished by introducing regulatory elements, in particular by the introducing a promoter capable of effecting transcription of an endogenous gene encoding the enzyme variant of the invention.

Method of Producing the Polypeptides

In another aspect the present invention provides a method of producing an isolated enzyme variant of the invention. In the method of the invention, a suitable production cell is genetically engineered by the introduction of exogenous polynucleotides to allow for expression of the enzyme variant, and the cell is cultured under conditions permitting the production of the polypeptide, followed by recovery of the desired polypeptide.

The polynucleotide of the invention may be incorporated into a desired production or host cell by methods known in the art, e.g. those described by Sambrook et al. [Sambrook et al.: Molecular Cloning: A Laboratory Manual, Cold Spring Harbor Lab., Cold Spring Harbor, N.Y. 1989]. Any technique that facilitates the introduction of exogenous polynucleotides into the desired cell may be employed, including methods like transduction, transfection, transformation, infection, etc.

The polynucleotide of the invention may in particular be obtained by site directed mutagenesis, or even by random mutagenesis.

The polynucleotide of the invention may be derived from any suitable source. The polynucleotide of the invention preferably is derived from an insect or a lower vertebrate. In a more preferred embodiment, which the polynucleotide of the invention is derived from, or produced on the basis of on the basis of any publically available cDNA library.

In a preferred embodiment the polynucleotide of the invention may be obtained using the PCR primers described in the working examples and presented as SEQ ID NOS: 7-8 and 13-20.

The isolated polynucleotide of the invention may be obtained by methods known in the art, e.g. those described in the working examples below.

Biological Activity

In contrast to most known deoxyribonucleoside kinases that have distinct, although partially overlapping substrate specificities and efficiencies, the deoxyribonucleoside kinase variants of the invention show increased relative efficiencies towards different substrates when compared to the wild-type enzyme.

In a preferred embodiment the ratio “k_{cat}/Kₘ(substrate)/k_{cat}/Kₘ(nucleoside analogue)” (i.e. the ratio between on the one side “k_{cat}/Kₘ” for at least one native substrate, and on the other side “k_{cat}/Kₘ” for at least one nucleoside analogue) is decreased by at least at least 5 fold, more preferred at least 10 fold, most preferred at least 20 fold.

As defined herein a kinase enzyme variant is considered to have increased sensitivity if its phosphorylating activity increases more than one fold over the wild-type (parent) enzyme in respect of one or more of its substrates.

In a preferred embodiment the different substrate is a nucleoside analogue. Preferred nucleoside analogues include aciclovir (9-[2-hydroxy-ethoxy]-methyl-guanosine), buciclovir, famciclovir, ganciclovir (9-[2-hydroxy-1-(hydroxymethyl)ethoxyl-methyl]-guanosine), penciclovir, valciclovir, trifluorothymidine, AZT (3′-azido-3′-thymidine), AIU (5′-iodo-5′-amino-2′,5′-dideoxyuridine), ara-A (adenosine-arabinoside; Vivarabine), ara-C (cytidine-arabinoside)i ara-G (9-beta-D-arabinofuranosylguanine), ara-T, 1-beta-D-arabinofuranosyl thymine, 5-ethyl-2′-deoxyuridine, 5-iodo-5′-amino-2,5′-dideoxyuridine, 1-[2-deoxy-2-fluoro-beta-D-arabino furanosyl]-5-iodouracil, idoxuridine (5-iodo-2′deoxyuridine), fludarabine (2-Fluoroadenine 9-beta-D-Arabinofuranoside), gencitabine, 2′,3′-dideoxyinosine (ddI), 2′,3′-dideoxycytidine (ddC), 2′,3′-dideoxythymidine (ddT), 2′,3′-dideoxyadenosine (ddA), 2′,3′-dideoxyguanosine (ddG), 2-chloro-2′-deoxyadenosine (2CdA), 5-fluorodeoxyuridine, BVaraU ((E)-5-(2-bromovinyl)-1-beta-D-arabinofuranosyluracil), BVDU (5-bromovinyl-deoxyuridine), FIAU (1-(2-deoxy-2-fluoro-beta-D-arabinofuranosyl)-5-iodouracil), 3TC (2′-deoxy-3′-thiacytidine), dFdC gemcitabine (2′,2′-difluorodeoxycytidine), dFdG gemcitabine (2′,2′-difluorodeoxyguanosine), or d4T (2′,3′didehydro-3′-deoxythymidine).

Gene therapy has recently emerged as a new method of therapeutic intervention to treat various cancers. In addition this approach can be used to combat viral infections and has applications in transplantation technology. The basis of this therapy is that a kinase gene is introduced into target cells where the gene will be expressed. The introduced kinase can then specifically activate otherwise harmless pro-drugs, which in the activated form are toxic and either will lead to cell death or inhibition of virus replication.

Deoxynucleoside analogues like AZT (Zidovudine, Retrovir®), ddC (Zalcitabine, Hivid®) or AraC (Cytarabine) are widely used to treat cancer and virus infected patients. In target cells these pro-drugs must be anabolised to their triphosphate form to become toxic and lead to cell death or to inhibit virus replication. The rate-limiting step in this activation process is the phosphorylation to the nucleoside monophosphate. However, phosphorylation of many nucleoside analogues is often inefficient in the target cells, or it occurs also un-specifically in non-target cells.

The efficacy and selectivity of these drugs can-be greatly improved using the pro-drug-activating genes coding for the deoxynucleoside kinase variants of the present invention.

So, viewed from one aspect the invention provides methods of sensitising cells to prodrugs, which method comprises the steps of

(i) transfecting said cell with a polynucleotide sequence of the invention encoding an enzyme that promotes the conversion of said prodrug into a (cytotoxic) drug; and

(ii) delivering said prodrug to said cell;

wherein said cell is more sensitive to said (cytotoxic) drug than to said prodrug.

In a preferred embodiment of this aspect the prodrug is a nucleoside analogue. In a more preferred embodiment, the nucleoside analogue is aciclovir (9-[2-hydroxy-ethoxy]-methyl-guanosine), buciclovir, famciclovir, ganciclovir (9-[2-hydroxy-1-(hydroxymethyl)ethoxyl-methyl]-guanosine), penciclovir, valciclovir, trifluorothymidine, AZT (3′-azido-3′-thymidine), AIU (5′-iodo-5′-amino-2′,5′-dideoxyuridine), ara-A (adenosine-arabinoside; Vivarabine), ara-C (cytidine-arabinoside), ara-G (9-beta-D-arabinofuranosylguanine), ara-T, 1-beta-D-arabinofuranosyl thymine, 5-ethyl-2′-deoxyuridine, 5-iodo-5′-amino-2,5′-dideoxyuridine, 1-[2-deoxy-2-fluoro-beta-D-arabino furanosyl]-5-iodouracil, idoxuridine (5-iodo-2′deoxyuridine), fludarabine (2-Fluoroadenine 9-beta-D-Arabinofuranoside), gencitabine, 2′,3′-dideoxyinosine (ddI), 2′,3′-dideoxycytidine (ddC), 2′,3′-dideoxythymidine (ddT), 2′,3′-dideoxyadenosine (ddA), 2′,3′-dideoxyguanosine (ddG), 2-chloro-2′-deoxyadenosine (2CdA), 5-fluorodeoxyuridine, BVaraU ((E)-5-(2-bromovinyl)-1-beta-D-arabinofuranosyluracil), BVDU (5-bromovinyl-deoxyuridine), FIAU (1-(2-deoxy-2-fluoro-beta-D-arabinofuranosyl)-5-iodouracil), 3TC (2′-deoxy-3′-thiacytidine), dFdC gemcitabine (2′,2′-difluorodeoxycytidine), dFdG (2′,2′-difluorodeoxyguanosine), or d4T (2′,3′didehydro-3′-deoxythymidine).

Viewed from another aspect the invention provides means and methods for combating pathogenic agents in a subject, which subject may in particular be a warm-blooded animal including a human.

In a preferred embodiment the invention provides a method of inhibiting a pathogenic agent in a warm-blooded animal, which method comprises administering to said animal a polynucleotide sequence of the invention, or a vector of the invention.

In a more preferred embodiment, the polynucleotide sequence or said vector is administered in vivo.

In another preferred embodiment, the pathogenic agent is a virus, a bacteria or a parasite.

In yet another preferred embodiment, the pathogenic agent is a tumour cell, or an autoreactive immune cell.

The method of the invention for inhibiting a pathogenic agent in a warm-blooded animal further comprising the step of administering a nucleoside analogue to said warm-blooded animal.

In a preferred embodiment the nucleoside analogue is aciclovir (9-[2-hydroxy-ethoxy]-methyl-guanosine), buciclovir, famciclovir, ganciclovir (9-[2-hydroxy-1-(hydroxymethyl)ethoxyl-methyl]-guanosine), penciclovir, valciclovir, trifluorothymidine, AZT (3′-azido-3′-thymidine), AIU (5′-iodo-5′-amino-2′,5′-dideoxyuridine), ara-A (adenosine-arabinoside; Vivarabine), ara-C (cytidine-arabinoside), ara-G (9-beta-D-arabinofuranosylguanine), ara-T, 1-beta-D-arabinofuranosyl thymine, 5-ethyl-2′-deoxyuridine, 5-iodo-5′-amino-2,5′-dideoxyuridine, 1-[2-deoxy-2-fluoro-beta-D-arabino furanosyl]-5-iodouracil, idoxuridine (5-iodo-2′deoxyuridine), fludarabine (2-Fluoroadenine 9-beta-D-Arabinofuranoside), gencitabine, 2′,3′-dideoxyinosine (ddI), 2′,3′-dideoxycytidine (ddC), 2′,3′-dideoxythymidine (ddT), 2′,3′-dideoxyadenosine (ddA), 2′,3′-dideoxyguanosine (ddG), 2-chloro-2′-deoxyadenosine (2CdA), 5-fluorodeoxyuridine, BVaraU ((E)-5-(2-bromovinyl)-1-beta-D-arabinofuranosyluracil), BVDU (5-bromovinyl-deoxyuridine), FIAU (1-(2-deoxy-2-fluoro-beta-D-arabinofuranosyl)-5-iodouracil), 3TC (2′-deoxy-3′-thiacytidine), dFdC gemcitabine (2,2′-difluorodeoxycytidine), dFdG (2′,2′-difluorodeoxyguanosine), or d4T (2′,3′didehydro-3′-deoxythymidine).

Pharmaceutical Compositions

In another aspect the invention provides novel pharmaceutical compositions comprising a therapeutically effective amount of the enzyme variant of the invention.

For use in therapy the enzyme variant of the invention may be administered in any convenient form. In a preferred embodiment, the enzyme variant of the invention is incorporated into a pharmaceutical composition together with one or more adjuvants, excipients, carriers and/or diluents, and the pharmaceutical composition prepared by the skilled person using conventional methods known in the art.

Such pharmaceutical compositions may comprise the enzyme variant of the invention, or antibodies hereof. The composition may be administered alone or in combination with one or more other agents, drugs or hormones.

The pharmaceutical composition of this invention may be administered by any suitable route, including, but not limited to oral, intravenous, intramuscular, interarterial, intramedullary, intrathecal, intraventricular, transdermal, subcutaneous, intraperitoneal, intranasal, anteral, topical, sublingual or rectal application, buccal, vaginal, intraorbital, intracerebral, intracranial, intraspinal, intraventricular, intracistemal, intracapsular, intrapulmonary, transmucosal, or via inhalation.

Further details on techniques for formulation and administration may be found in the latest edition of Remington's Pharmaceutical Sciences (Maack Publishing Co., Easton, Pa.).

The active ingredient may be administered in one or several doses per day. Currently contemplated appropriate dosages are between 0.5 ng enzyme variant/kg body weight to about 50 μg/kg per administration, and from about 1.0 μg/kg to about 100 μg/kg daily.

The dose administered must of course be carefully adjusted to the age, weight and condition of the individual being treated, as well as the route of administration, dosage form and regimen, and the result desired, and the exact dosage should of course be determined by the practitioner.

In further embodiments, the enzyme variant of the invention may be administered by genetic delivery, using cell lines and vectors as described below under methods of treatment. To generate such therapeutic cell lines, the polynucleotide of the invention may be inserted into an expression vector, e.g. a plasmid, virus or other expression vehicle, and operatively linked to expression control sequences by ligation in a way that expression of the coding sequence is achieved under conditions compatible with the expression control sequences. Suitable expression control sequences include promoters, enhancers, transcription terminators, start codons, splicing signals for introns, and stop codons, all maintained in the correct reading frame of the polynucleotide of the invention so as to permit proper translation of mRNA. Expression control sequences may also include additional components such as leader sequences and fusion partner sequences.

Methods of Treatment

The present invention, which relates to polynucleotides and proteins, polypeptides, peptide fragments or derivatives produced therefrom, as well as to antibodies directed against such proteins, peptides or derivatives, may be used for treating or alleviating a disorder or disease of a living animal body, including a human, which disorder or disease is responsive to the activity of a cytotoxic agent.

The disorder, disease or condition may in particular be a cancer or a viral infection.

The enzyme variants of the present invention may be used directly via, e.g., injected, implanted or ingested pharmaceutical compositions to treat a pathological process responsive to the enzyme variant.

The polynucleotide of the invention, including the complementary sequences thereof, may be used for the expression of the enzyme variant of the invention. This may be achieved by cell lines expressing such proteins, peptides or derivatives of the invention, or by virus vectors encoding such proteins, peptides or derivatives of the invention, or by host cells expressing such proteins, peptides or derivatives. These cells, vectors and compositions may be administered to treatment target areas to affect a disease process responsive to cytotoxic agents.

Suitable expression vectors may be derived from lentiviruses, retroviruses, adenoviruses, herpes or vaccinia viruses, or from various bacterially produced plasmids, and may be used for in vivo delivery of nucleotide sequences to a whole organism or a target organ, tissue or cell population. Other methods include, but are not limited to, liposome transfection, electroporation, transfection with carrier peptides containing nuclear or other localising signals, and gene delivery via slow-release systems. In still another aspect of the invention, “antisense” nucleotide sequences complementary to the nucleotide of the invention or portions thereof, may be used to inhibit or enhance enzyme variant expression.

In yet another aspect the invention relates to a method of treating or alleviating a disorder, disease or condition of a living animal body, including a human, which disorder or disease is responsive to the activity of cytotoxic agents.

### BRIEF DESCRIPTION OF THE DRAWINGS

The present invention is further illustrated by reference to the accompanying drawing, in which:

FIG. 1 shows the influence of the nucleotide analogue concentrations [PTP or 8-oxo-dGTP; 2.5, 5.0, 10.0, 20.0, 50.0, 100.0 and 200.0 μM, respectively] in the mutagenic PCR on TK activity [relative number of colonies on TK selection plates (0-60%)]; and

FIGS. 2A-D show the kinetic patterns for the inhibition of thymidine phosphorylation by TTP. Initial velocities of rDm-dNK (FIG. 2A) and rMuD (FIG. 2B) are showed as a function of varied dThd at fixed TTP concentrations. Double-reciprocal plots of the same data (FIG. 2C for rDmdNK; and FIG. 2D for rMuD) demonstrate the type of inhibition. [FIGS. 2A and 2C: O 0 μM TTP, □ 9.8 μM TTP, Δ 29.3 μM TTP, ∇ 48.9 μM TTP; FIGS. 2B and 2D: O 0 μM TTP, □ 500 μM TTP, Δ 1000 μM TTP, ∇ 2000 μM TTP]. The solid lines represents the best fits of the equations calculated as described in Example 2 (Analysis of Kinetic Data).

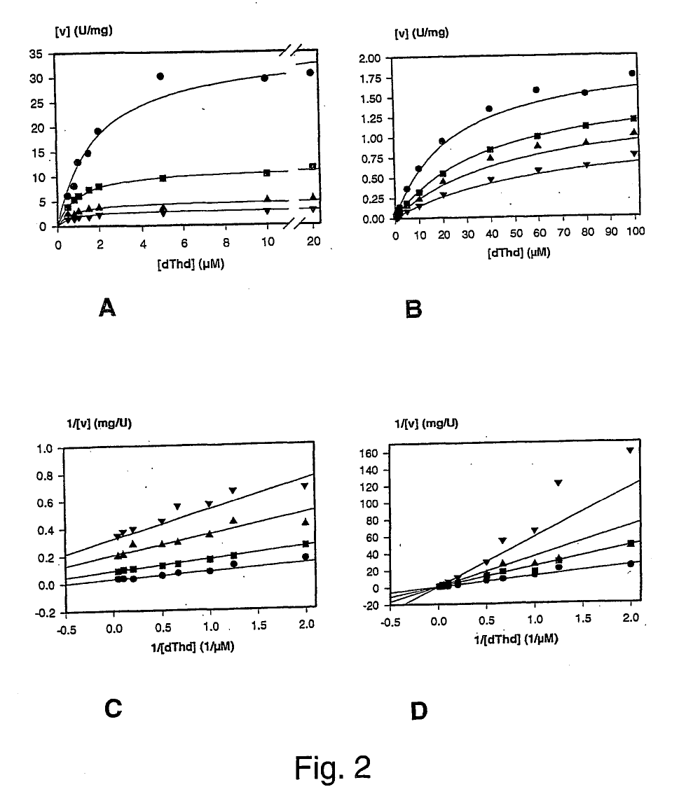

### EXAMPLES

The invention is further illustrated with reference to the following examples which are not intended to be in any way limiting to the scope of the invention as claimed.

### Example 1

### PCR Induced Dm-dNK Variants

A directed evolution approach, based on mutagenic PCR, was employed to generate mutant kinase forms. The open reading frame (ORF) for Dm-dNK was mutagenized using different nucleotide analogue concentrations and the influence of the different nucleotide analogue concentrations was investigated. The mutagenized PCR fragments were ligated into an expression plasmid and subsequently transformed into the TK deficient E. coli strain KY895.

Random Mutagenesis and Mutant Library Construction

The expression-vector pGEX-2T-rDm-dNK [Munch-Petersen et al., J. Biol. Chem. 2000 275 (9) 6673-6679] was used as template for PCR mutagenesis.

The open reading frame (ORF) for Dm-dNK was amplified using the following primers:

Dm-TK3: 5′-CGCGGATCCATGGCGGAGGCAGCATCCT-3′ (SEQ ID NO: 7); and

Dm-TK4: 5′-CGGAATTCTTATCTGGCGACCCTCTGGCGT-3′ (SEQ ID NO: 8).

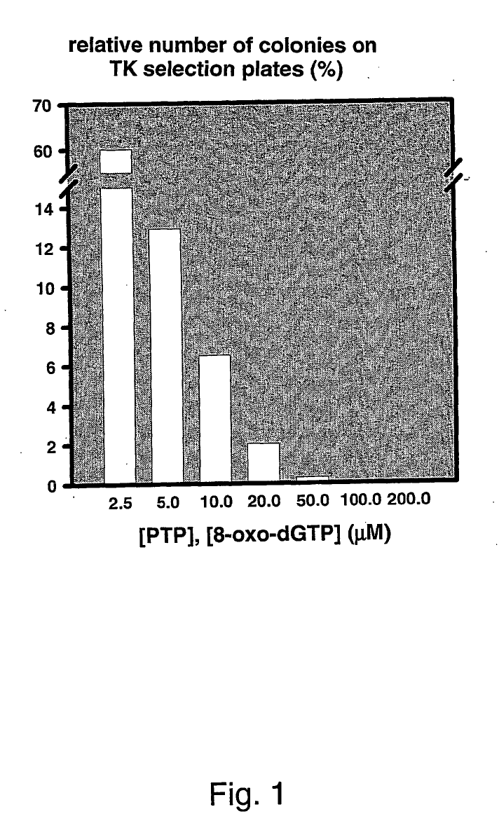

PCR was done in 2 steps. The first PCR was done in 20 μl reactions with 0.15 units Taq Polymerase from Amersham Corp. in the supplied buffer. Template DNA 10 fmol, primers with 20 pmol each, dNTPs at 0.2 mM each were used. The nucleotide analogues 6-(2-deoxy-β-D-erythropentofuranosyl)-3,4-dihydro-8H-pyrimido-[4,5C][1,2]oxazine-7-one-5′-triphosphate (dPTP) and 2′-Deoxy-8-hydroxyguanosine-5′-triphosphate (8-oxo-dGTP), both available from Amersham Corp., were present at concentrations as shown in FIG. 1.

PCR conditions were: Denaturation at 94° C. for 5 minutes, 25 cycles with 94° C. for 45 seconds, 50° C. for 45 seconds, 72° C. for 2 minutes and finally prolongation at 72° C. for 10 minutes.

The PCR products were purified with the PCR purification kit from Boehringer Mannhein and eluted in 80 μl of 5 mM Tris/HCl pH 7.5. 40 μl of this eluate was used in the second PCR without nucleotide analogues, which was done in a volume of 65 μl with 0.5 units Taq Polymerase, 65 pmol of each primer, 0.2 mM of each dNTP. PCR conditions were the same as in the first PCR, but cycling was done for 15 cycles only.

The mutagenized PCR fragments were purified by the PCR Kit from Boehringer Mannhein, cut with BamHI and EcoRI and sub-cloned into the multiple cloning site of the pGEX-2T plasmid vector. The TK deficient E. coli strain KY895 [F tdk-1 ilv] [Igarashi K, Hiraga S & Yura T: A deoxythymidine kinase deficient mutant of Eschericha coli. II. Mapping and transduction studies with phage Φ80; Genetics 1967 57 643-6541, was electro-transformed with the ligation mix, using standard techniques, and plated on LB-ampicillin (100 μg/ml) plates.

The relative number of colonies carrying re-circularised vector was determined by colony PCR of randomly picked clones.

Degree of Mutagenicity

The influence of different nucleotide analogue concentrations in the mutagenic PCR was investigated. The degree of mutagenicity was evaluated as the loss of TK activity. This was done by replica plating of at least 500 colonies from LB-ampicillin plates to TK selection plates [Black M E, Newcomb T G, Wilson H M P & Loeb L A: Creation of drug-specific herpes simplex virus type 1 thymidine kinase mutants for gene therapy; Proc. Natl. Acad. Sci. USA 1996 93 3525-3529] and counting the number of colonies surviving on the TK selection plates. Results were corrected for the re-circularisation of the vector.

Selection of Mutants

First, colonies were selected for restored TK activity by replica plating them on TK selection plates [Black M E, Newcomb T G, Wilson H M P & Loeb L A: Creation of drug-specific herpes simplex virus type 1 thymidine kinase mutants for gene therapy; Proc. Natl. Acad. Sci. USA 1996 93 3525-3529]. Only mutants complementing the TK negative E. coli strain KY895 give rise to colonies on this selection medium.

Overnight cultures of these colonies were diluted 200-fold in 10% glycerol and 2 μl drops of the dilution were spotted on M9 minimal medium plates [Ausubel F, Brent R, Kingston R E, Moore D D, Seidman J G, Smith J A & Struhl K (Eds.): Short protocols in molecular biology; Wiley, USA, 3^{rd} Edition, 1995, p.1-2] supplemented with 0.2% glucose, 40 μg/ml isoleucine, 40 μg/ml valine, 100 μg/ml ampicillin and with or without nucleoside analogues.

For the first screening 200 μl of 2.5 mM AraC, 500 μM AZT, 500 μM ddA or 25 mM ddC were evenly spread on the surface of a 10 ml solidified medium containing 8.5 cm diameter plate. Growth of colonies was visually inspected after 24 hours at 37° C. From clones not growing on nucleoside analogue containing plates, but growing normally on plates without the nucleoside analogue, the plasmid was isolated and re-transformed into KY895. These clones were re-tested to verify the plasmid born phenotype.

### Example 2

### Characterisation of the Enzyme Variants

Sequencing

Sequencing by the Sanger dideoxynucleotide method was performed manually, using the Thermo Sequenase radio-labelled terminator cycle sequencing kit and P³³ labelled ddNTPs (Amersham Corp.) on the purified plasmids.

Determination of LD₁₀₀ (in vivo Characterisation)

All clones with increased sensitivity towards at least one nucleoside analogue were tested on M9 plates with logarithmic dilution of the nucleoside analogues to determine the lethal dose (LD₁₀₀) of the nucleoside analogues, at which no growths of bacteria could be seen. Plates with the concentration ranges 10-1000 μM of AraA, 3.16-1000 μM of AraC; 0.01-100 μM of AZT; 0.316-31.6 μM of ddA; 0.0316-100 μM of 2CdA or 10-3500 μM of ddC; were used to determine the LD₁₀₀ (the concentrations which cause 100% lethality) of putative mutants.

Plates were prepared by mixing the medium with the analogues at the lowest temperature possible, before pouring the plates.

The results of these tests are presented in Table 2, below.

TABLE 2
LD₁₀₀

| Amino | | | | 1 | 1 | 1 | 1 | 1 | 1 | 1 | 1 | 1 | 2 | 2 | 2 | 2 | 2 | 2 | 2 | 2 | 2 | 3 | 3 | 3 | 3 | 3 | 3 | 3 | 3 | | | | ddC |
|---|---|---|---|---|---|---|---|---|---|---|---|---|---|---|---|---|---|---|---|---|---|---|---|---|---|---|---|---|---|---|---|---|---|
| acid | 5 | 6 | 9 | 0 | 0 | 1 | 3 | 3 | 3 | 6 | 6 | 9 | 1 | 1 | 1 | 2 | 2 | 2 | 2 | 7 | 8 | 0 | 0 | 1 | 1 | 2 | 3 | 4 | 5 | LD₁₀₀ | (μM) | | 2CdA |
| position | 1 | 2 | 1 | 0 | 2 | 4 | 4 | 8 | 9 | 7 | 8 | 9 | 4 | 6 | 7 | 0 | 2 | 8 | 9 | 7 | 1 | 7 | 9 | 6 | 8 | 1 | 4 | 7 | 2 | araC | AZT | ddA | araA |
| Dm-dNK | M | T | N | N | I | N | N | L | M | V | T | I | V | N | M | N | S | Y | N | V | Y | S | K | D | N | N | F | L | K | 1000 | 100 | 31.6 | >3500 |
| | | | | | | | | | | | | | | | | | | | | | | | | | | | | | | | | | 100 |
| | | | | | | | | | | | | | | | | | | | | | | | | | | | | | | | | | >1000 |
| MuA | | | | | | | | | | | | M | | S | V | | | | | | | | | N | | | | | | 31.6 | 3.16 | 316 | |
| | | | | | | | | | | | | | | | | | | | | | | | | | | | | | | | | | 31.6 |
| | | | | | | | | | | | | | | | | | | | | | | | | | | | | | | | | | >1000 |
| MuB | | | | | | | | | | | | | | | | | | | | | | | | | D | | | P | | 100 | 31.6 | 31.6 | 1000 |
| | | | | | | | | | | | | | | | | | | | | | | | | | | | | | | | | | 100 |
| | | | | | | | | | | | | | | | | | | | | | | | | | | | | | | | | | >1000 |
| MuC | | | | | | | | | | A | | | | | | | | | | | | | | | | | | | | 100 | 100 | 31.6 | 1000 |
| | | | | | | | | | | | | | | | | | | | | | | | | | | | | | | | | | 100 |
| | | | | | | | | | | | | | | | | | | | | | | | | | | | | | | | | | >1000 |
| MuD | | | | D | | | D | | | | | | | | | | | | | | | | | | | | | | | 316 | 0.316 | 10 | 316 |
| | | | | | | | | | | | | | | | | | | | | | | | | | | | | | | | | | 100 |
| | | | | | | | | | | | | | | | | | | | | | | | | | | | | | | | | | >1000 |
| MuE | | A | | | | | | | | A | | | | | | | | | | | | | | | | S | | | | 100 | 100 | 31.6 | >3500 |
| | | | | | | | | | | | | | | | | | | | | | | | | | | | | | | | | | 100 |
| | | | | | | | | | | | | | | | | | | | | | | | | | | | | | | | | | >1000 |
| MuF | | | | | T | | | | | | | | | | | | | | | | | | | | D | | | | | 100 | 31.6 | 31.6 | >3500 |
| | | | | | | | | | | | | | | | | | | | | | | | | | | | | | | | | | 100 |
| | | | | | | | | | | | | | | | | | | | | | | | | | | | | | | | | | >1000 |
| MuG | | | | | | D | | | | | | | | | V | | | | | | H | | | | | | | | | 316 | 10 | 31.6 | >3500 |
| | | | | | | | | | | | | | | | | | | | | | | | | | | | | | | | | | 100 |
| | | | | | | | | | | | | | | | | | | | | | | | | | | | | | | | | | >1000 |
| MuH | | | | | | | | | | | A | | | | | | | | | | | | | | | | | | | 31.6 | 100 | 31.6 | 316 |
| | | | | | | | | | | | | | | | | | | | | | | | | | | | | | | | | | 10 |
| | | | | | | | | | | | | | | | | | | | | | | | | | | | | | | | | | >1000 |
| MuI | | | | | | | | | | | | | | | | | | C | | A | | | R | | | | | | | 1000 | 10 | 31.6 | >3500 |
| | | | | | | | | | | | | | | | | | | | | | | | | | | | | | | | | | 100 |
| | | | | | | | | | | | | | | | | | | | | | | | | | | | | | | | | | >1000 |
| MuJ | T | | | | | | | | | | A | | | | | S | | | | | | | | | | | | | | 100 | 100 | 31.6 | 1000 |
| | | | | | | | | | | | | | | | | | | | | | | | | | | | | | | | | | 100 |
| | | | | | | | | | | | | | | | | | | | | | | | | | | | | | | | | | >1000 |
| MuK | | | | | | | | | | | | | | | | | W | | | | | | | | | | L | | | >1000 | 10 | 31.6 | >3500 |
| | | | | | | | | | | | | | | | | | | | | | | | | | | | | | | | | | 100 |
| | | | | | | | | | | | | | | | | | | | | | | | | | | | | | | | | | >1000 |
| MuL | | | D | | | | D | | | | | | | | | | | | | | | | | | | | | | | >1000 | 3.16 | 31.6 | >3500 |
| | | | | | | | | | | | | | | | | | | | | | | | | | | | | | | | | | 100 |
| | | | | | | | | | | | | | | | | | | | | | | | | | | | | | | | | | >1000 |
| MuM | | | | | | | S | S | L | | | | | | | | | | | | | | | | | | | | N | >1000 | 10 | 31.6 | >3500 |
| | | | | | | | | | | | | | | | | | | | | | | | | | | | | | | | | | 100 |
| | | | | | | | | | | | | | | | | | | | | | | | | | | | | | | | | | >1000 |
| M15 + 133 | | | | | | | | | | | A | M | | S | V | | | | | | | | | N | | | | | | 31.6 | 10 | 3.16 | 316 |
| | | | | | | | | | | | | | | | | | | | | | | | | | | | | | | | | | 10 |
| | | | | | | | | | | | | | | | | | | | | | | | | | | | | | | | | | >1000 |
| M15 + 44 | | | | D | | | D | | | | | M | | S | V | | | | | | | | | N | | | | | | 1000 | 1 | 3.16 | 1000 |
| | | | | | | | | | | | | | | | | | | | | | | | | | | | | | | | | | 100 |
| | | | | | | | | | | | | | | | | | | | | | | | | | | | | | | | | | >1000 |
| B2 | | | | | | | | | | A | | M | | S | V | | | | | | | | | N | | | | | | 10 | 3.16 | 0.316 | 100 |
| | | | | | | | | | | | | | | | | | | | | | | | | | | | | | | | | | 3.16 |
| | | | | | | | | | | | | | | | | | | | | | | | | | | | | | | | | | 1000 |
| B5 | | | | | | | | | | A | | | | | | | | | | | | | | | D | | | P | | 10 | 31.6 | 10 | 316 |
| | | | | | | | | | | | | | | | | | | | | | | | | | | | | | | | | | 1 |
| | | | | | | | | | | | | | | | | | | | | | | | | | | | | | | | | | 100 |
| B10 | | | | D | | | D | | | | | | | | | | | | | | | | | | D | | | P | | 316 | 0.316 | 1 | 316 |
| | | | | | | | | | | | | | | | | | | | | | | | | | | | | | | | | | 31.6 |
| | | | | | | | | | | | | | | | | | | | | | | | | | | | | | | | | | >1000 |
| B15 | | | | | | | | | | | A | | | | | | | | | | | | | | D | | | P | | 3.16 | 31.6 | 10 | 316 |
| | | | | | | | | | | | | | | | | | | | | | | | | | | | | | | | | | <1 |
| | | | | | | | | | | | | | | | | | | | | | | | | | | | | | | | | | 100 |
| B17 | | | | | | | | | V | | | | | | | | | | | | | | | | D | | | P | | 31.6 | 31.6 | 10 | 316 |
| | | | | | | | | | | | | | | | | | | | | | | | | | | | | | | | | | 3.16 |
| | | | | | | | | | | | | | | | | | | | | | | | | | | | | | | | | | >1000 |
| F2 | | | | | | | | | | | | M | | S | V | | | | S | | | P | | N | | | | | | 31.6 | 10 | 10 | 316 |
| | | | | | | | | | | | | | | | | | | | | | | | | | | | | | | | | | 10 |
| | | | | | | | | | | | | | | | | | | | | | | | | | | | | | | | | | >1000 |
| F7 | | | | | | | | | | | | M | A | S | V | | | | | | | | | N | | | | | | 100 | 100 | 10 | >1000 |
| | | | | | | | | | | | | | | | | | | | | | | | | | | | | | | | | | 10 |
| | | | | | | | | | | | | | | | | | | | | | | | | | | | | | | | | | >1000 |

Protein Expression and Purification (in vitro Characterisation)

Higher expression was obtained in E. coli strain BL21 (Pharmacia Biotech, Sweden) than in KY895 cells. Expression and purification of thrombin cleaved recombinant wild-type Dm-dNK or mutant MuD was done as described by Munch-Petersen et al. [J. Biol. Chem. 2000 275 (9) 6673-6679]. Purified proteins are referred to as rDm-dNK or rMuD.

Enzyme Assays

Nucleoside kinase activities were determined by initial velocity measurements based on four time samples by the DE-81 filter paper assay using tritium-labelled substrates. Alternatively ADP production was measured by a spectrometric assay. Both assays were done as described by Munch-Petersen et al. [J. Biol. Chem. 2000 275 (9) 6673-6679].

Analysis of Kinetic Data

Kinetic data were evaluated as described in Knecht et al. [Knecht W, Bergjohann U, Gonski S, Kirschbaum B, Loffler M: Functional expression of a fragment of human dihydroorotate dehydrogenase by means of the baculovirus expression vector system, and kinetic investigation of the purified recombinant enzyme; Eur. J. Biochem. 1996 240 (1) 292-301] by non-linear regression analysis using the Michaelis-Menten equation v=Vₘₐₓ×[S]/(Kₘ+[S]).

Concentrations giving 50% inhibition of enzyme activity (IC₅₀) were determined by fitting the equation v_{I}=v₀/(1+[I]/IC₅₀) to the velocities of the reaction in the presence of varying inhibitor concentrations [I]. v_{I} and v₀ are the velocities in presence or absence of inhibitor, respectively.

To determine the type of inhibition, Vₘₐₓ and Kₘ values were determined at 3 different inhibitor concentrations. Deviations of Vₘₐₓ and Kₘ values in comparison with the constants for the non-inhibited enzymatic reaction were considered to determine whether the inhibition was competitive, un-competitive or non-competitive.

Once an inhibition pattern was established, the unchanged equation for non-competitive inhibition v=Vₘₐₓ×[S]/{Kₘ×(1+[I]/K_{ic})+(1+[I]/Kᵢᵤ)×[S]} was fit the entire data set. K_{ic} is the competitive inhibition constant, Kᵢᵤ is the un-competitive inhibition constant (Liebecg C: IUBMB Biochemical nomenclature and related documents; Portland Press, London, 1992].

### Example 3

### Sequence Determination

The Basic local alignment search tool (BLAST) was used to search the publically available expressed sequence tag (EST) libraries in the GenBank database at the National Institute for Biotechnology information and to identify ESTs that encode enzymes similar to Dm-dNK (GenBanK ACCN AF226281). In this way the ESTs ACCN AU004911 from Bombyx mori and ACCN AW159435 from Xenopus laevis were identified.

The ESTs were obtained from the Genome Research Group, National Institute of Radiological Sciences, Anagawa 4-9-1, Inage, Chiba 263-8555, Japan (ACCN AU004911) and from Lita Annenberg Hazen Genome Sequencing Center, Cold Spring Harbor Laboratory, PO Box 100, Cold Spring Harbor, N.Y. 11724, USA (AW159435). The complete open reading frame of the deoxyribonucleoside kinases encoded by these two ESTs was determined by DNA sequencing (see Example 2).

The complete open reading frames were then submitted to GenBank and received assignments ACCN AF226281 (Bombyx mori deoxyribonucleoside kinase, presented as SEQ ID NO: 3) and ACCN AF250861 (Xenopus laevis deoxyribonucleoside kinase, presented as SEQ ID NO: 5).

### Example 4

### Hybrid Enzymes

This example described the construction of hybrid enzymes in the expression vector pGEX-2T (pGEX-2T-rdmk/bmk and pGEX-2T-rbmk/dmk, respectively).

The expression plasmid pGEX-2T-rBm-dNK was constructed essentially as described by Munch-Petersen et al. [Munch-Petersen et al., J. Biol. Chem. 2000 275 (9) 6673-6679] for pGEX-2T-rDm-dNK using the primers Bm_{for}1 and Bmᵣₑᵥ1, and the cDNA for Bombyx mori kinase, obtained as described in Example 3, as template.

The following 1^{th} PCR's were done:

| | bmk/dmk 1 | bmk/dmk 2 | dmk/bmk 1 | dmk/bmk 2 |
|---|---|---|---|---|
| Primer 1 | pGEX-2T_{for} | pGEX-2Tᵣₑᵥ | PGEX-2T_{for} | pGEX-2Tᵣₑᵥ |
| Primer 2 | bmk-Nₜₑᵣₘ | dmk-Cₜₑᵣₘ | dmk-Nₜₑᵣₘ | bmk-Cₜₑᵣₘ |
| Template | pGEX-2T- | pGEX-2T- | pGEX-2T- | pGEX-2T- |
| | rBm-dNK | rDm-dNK | rDm-dNK | rDm-dNK |

The PCR conditions were: Denaturation at 94° C. for 5 minutes, 30 times cycling at 94° C. for 1 minute, 50° C. for 1 minute and 72° C. for 1 minute, and final prolongation for 10 minutes at 72° C.

The resulting fragments from all four PCR's were purified by the PCR Purification Kit from Boehringer Mannheim.

Then the following 2^{nd} PCR's were done:

| | bmk/dmk | dmk/bmk |
|---|---|---|
| Primer 1 | Bm_{for}1 | Dm-TK3 (SEQ ID NO: 7) |
| Primer 2 | Dm-TK4 (SEQ ID NO: 8) | Bmᵣₑᵥ1 |
| Template | bmk/dmk 1 and | dmk/bmk 1 and |
| | bmk/dmk 2 from the 1^{th} PCR | dmk/bmk 2 |

The PCR conditions were: Denaturation at 94° C. for 5 minutes, 30 times cycling at 94° C. for 1 minute, 45° C. for 5 minutes and 72° C. for 1 minute, and final prolongation for 10 minutes at 72° C.

The resulting fragments were cut, purified and subcloned into the expression vector obtained as described under Example 1.

Primers

Dm-TK3 (SEQ ID NO: 7);

Dm-TK4 (SEQ ID NO: 8);

pGEX-2T_{for}: 5′-acg ttt ggt ggt ggc gac ca-3′ (SEQ ID NO: 13);

pGEX-2Tᵣₑᵥ: 5′-ctc cgg gag ctg cat gtg tc-3′ (SEQ ID NO: 14);

bmk-Nₜₑᵣₘ: 5′-cta aaa atg gag cgc tcc att agc ttt act gga gtt gg-3′ (SEQ ID NO: 15);

dmk-Cₜₑᵣₘ: 5′-cca gta aag cta atg gag cgc tcc att ttt agc gc-3′ (SEQ ID NO: 16);

dmk-Nₜₑᵣₘ: 5′-gaa taa tga tcg ctc cat tat ttt tag ctt ctt gt-3′ (SEQ ID NO: 17);

bmk-Cₜₑᵣₘ: 5′-aag cta aaa ata atg gag cga tca tta ttc agt gc-3′ (SEQ ID NO: 18);

Bm_{for}1: 5′-tat cgc gga tcc atg agt gcc aac aat gtt aaa cca ttc acc-3′ (SEQ ID NO: 19); and

Bmᵣₑᵥ1: 5′-ccg gaa ttc gtc gac tta taa gat cct cat gtg agg tgt gat ctt g-3′ (SEQ ID NO: 20).

1. An isolated, mutated polynucleotide encoding a multi-substrate deoxyribonucleoside kinase enzyme, which mutated polynucleotide, when compared to the non-mutated polynucleotide, and upon transformation into a bacterial or eukaryotic cell, decreases at least 4 fold the lethal dose (LD₁₀₀) of at least one nucleoside analogue.

2. The mutated polynucleotide of claim 1, wherein said nucleoside analogue is aciclovir (9-[2-hydroxy-ethoxy]-methyl-guanosine), buciclovir, famciclovir, ganciclovir (9-[2-hydroxy-1-(hydroxymethyl)ethoxyl-methyl]-guanosine), penciclovir, valciclovir, trifluorothymidine, AZT (3′-azido-3′-thymidine), AIU (5′-iodo-5′-amino-2′,5′-dideoxyuridine), ara-A (adenosine-arabinoside; Vivarabine), ara-C (cytidine-arabinoside), ara-G (9-beta-D-arabinofuranosylguanine), ara-T, 1-beta-D-arabinofuranosyl thymine, 5-ethyl-2′-deoxyuridine, 5-iodo-5′-amino-2,5′-dideoxyuridine, 1-[2-deoxy-2-fluoro-beta-D-arabino furanosyl]-5-iodouracil, idoxuridine (5-iodo-2′deoxyuridine), fludarabine (2-Fluoroadenine 9-beta-D-Arabinofuranoside), gencitabine, 2′,3′-ideoxyinosine (ddI), 2′,3′-dideoxycytidine (ddC), 2′,3′-dideoxythymidine (ddT), 2′,3′-dideoxyadenosine (ddA), 2′,3′-dideoxyguanosine (ddG), 2-chloro-2′-deoxyadenosine (2CdA), 5-fluorodeoxyuridine, BVaraU ((E)-5-(2-bromovinyl)-1-beta-D-arabinofuranosyluracil), BVDU (5-bromovinyl-deoxyuridine), FIAU (1-(2-deoxy-2-fluoro-beta-D-arabinofuranosyl)-5-iodouracil), 3TC (2′-deoxy-3′-thiacytidine), dFdC gemcitabine (2′,2′-difluorodeoxycytidine), dFdG (2′,2′-difluorodeoxyguanosine), or d4T (2′,3′didehydro-3′-deoxythymidine).

3. The mutated polynucleotide of claim 1, which mutated polynucleotide, decreases at least 4 fold the lethal dose (LD₁₀₀) of at least two different nucleoside analogues, which analogous are based on two different sugar moieties and two different base moieties.

4. An isolated deoxyribonucleoside kinase variant encoded by the polynucleotide of claims 1-3.

5. The enzyme variant of claim 4, which variant is altered with respect to
(i) The ratio “k_{cat}/Kₘ(substrate)/k_{cat/K}ₘ(nucleoside analogue)” is decreased by at least at least 5 fold; and/or
(ii) The feedback inhibition by NTP's and dNTPs, in particular TTP, is decreased by at least 1.5 fold, as determined by its IC₅₀ value using 2 or 10 μM thymidine (dThd) as a substrate;
when compared to the wild-type enzyme.

6. The enzyme variant of claim 4, which decreases at least 4 fold the lethal dose (LD₁₀₀) of at least two different nucleoside analogues, which analogous are based on two different sugar moieties and two different base moieties.

7. The enzyme variant of claim 4, which variant, when compared to the wild-type enzyme, has been mutated in
(i) in a non-motif and/or a non-conserved region; and/or
(ii) in only one motif and/or conserved region; and/or
(iii) in any conserved position;
the regions and positions being as defined in Table 1.

8. The enzyme variant of claim 4, which variant comprises a mutation (incl. substitutions, additions and deletions) at one or more of the following positions 51, 62, 82, 91, 100, 102, 107, 112, 114, 134, 138, 139, 140, 164, 167, 168, 171, 199, 202, 207;211, 213; 214, 216, 217, 220, 222, 228, 229, 274, 277, 281, 283; 284; 307, 309, 316, 318, 321, 334, 347, and 352 (dNK numbering).

9. The enzyme variant of claim 6, which variant comprises a substitution conservative to those of G80, N81, I82, G83, S84, G85, K86, T87, T88, E107, P108, V109, E110, K111, W112, Y140, Q164, E201, R202, S203, C210, Y211, C212, P258, R265, I266, R267, Q268, R269, A270, R271, E274, L279, L282, or L293 (dNK numbering).

10. The enzyme variant of claim 6, which variant comprises one or more of the following mutations M51T; T62A; N91D; N100D; I102T; N114D; N134D; N134S; L138S; M139L; M139V; V167A; V167S; V167M; T168A; M171R; I199M; A207D; V214A; N216S; M217V; N220S; S222W; Y228C; N229S; V277A; Y281H; S307P; K309R; D316N; N318D; N321S; F334L; L347P; and K352N (dNK numbering).

11. The enzyme variant of claim 8, which variant comprises
M51T/T168A/N220S;
T62A/V167A/N321S;
N91D/N134D;
N100D/N134D;
N100D/N134D/N318D/L347P;
N100D/N134D/I199M/N216S/M217V/D316N;
I102T/N318D;
N114D/M217V/Y281H;
N134S/L138S/M139L/K352N;
M139V/N318D/L347P;
V167A/M171R/A207D;
V167S/M171R/A207D;
V167A/I199M/N216S/M217V/D316N;
V167A/N318D/L347P;
T168A/N318D/L347P;
T168A/I199M/N216S/M217V/D316N;
M171R/A207D;
I199M/V214A/N216S/M217V/D316N;
I199M/N216S/M217V/N229S/S307P/D316N;
I199M/N216S/M217V/D316N;
S222W/F334L;
Y228C/V277A/K309R; or
N318D/L347P (dNK numbering).

12. The enzyme variant of any of claims 3-9, which variant is derived from a multi-substrate deoxyribonucleoside kinase.

13. The enzyme variant of any of claims 3-9, which variant is a deoxyribonucleoside kinase derived from a human thymidine kinase 2 (hu-TK2); or a human deoxyguanosine kinase (hu-dGK); or a human deoxycytidine kinase (hu-dCK); or a Herpes simplex virus thymidine kinase (HSV1-TK).

14. The enzyme variant of any of claims 3-9, which variant is derived from an insect multi-substrate deoxyribonucleoside kinase.

15. The enzyme variant of claim 14, which is a hybrid deoxyribonucleoside kinase derived from two or more insect multi-substrate deoxyribonucleoside kinases.

16. The enzyme variant of claim 15, which hybrid deoxyribonucleoside kinase comprises at least 5 consecutive amino acids derived from each insect multi-substrate deoxyribonucleoside kinases.

17. The enzyme variant of claim 14, which variant is a deoxyribonucleoside kinase derived from a Drosophila melanogaster deoxyribonucleoside kinase (Dm-dNK), or a Bombyx mori deoxyribonucleoside kinase (Bm-dNK), or a Xenopus laevis deoxyribonucleoside kinase (Xen-dNK), or an Anopheles gambia deoxyribonucleoside kinase.

18. The enzyme variant of claim 17 being
Dm-dNK/M51T;
Dm-dNK/M51T/T168A/N220S;
Dm-dNK/T62A;
Dm-dNK/T62A/V167A/N321S;
Dm-dNK/N91D;
Dm-dNK/N91D/N134D;
Dm-dNK/N100D;
Dm-dNK/N100D/N134D;
Dm-dNK/N100D/N134D/N318D/L347P;
Dm-dNK/N100D/N134D/I199M/N216S/M217V/D316N;
Dm-dNK/I102T;
Dm-dNK/I102T/N318D;
Dm-dNK/N114D;
Dm-dNK/N114D/M217V/Y281H;
Dm-dNK/N134D;
Dm-dNK/N134S;
Dm-dNK/N134S/L138S/M139L/K352N;
Dm-dNK/L138S;
Dm-dNK/M139L;
Dm-dNK/M139V;
Dm-dNK/M139V/N318D/L347P;
Dm-dNK/V167A;
Dm-dNK/V167A/I199M/N216S/M217V/D316N;
Dm-dNK/V167A/N318D/L347P;
Dm-dNK/V167A/M171R/A207D;
Dm-dNK/V167S/M171R/A207D;
Dm-dNK/T168A;
Dm-dNK/T168A/N318D/L347P;
Dm-dNK/T168A/I199M/N216S/M217V/D316N;
Dm-dNK/M171R/A207D;
Dm-dNK/I199M;
Dm-dNK/I199M/V214A/N216S/M217V/D316N;
Dm-dNK/I199M/N216S/M217V/D316N;
Dm-dNK/I199M/N216S/M217V/N229S/S307P/D316N;
Dm-dNK/V214A;
Dm-dNK/N216S;
Dm-dNK/M217V;
Dm-dNK/N220S;
Dm-dNK/S222W;
Dm-dNK/S222W/F334L;
Dm-dNK/Y228C;
Dm-dNK/Y228C/V277A/K309R;
Dm-dNK/N229S;
Dm-dNK/V277A;
Dm-dNK/Y281H;
Dm-dNK/S307P;
Dm-dNK/K309R;
Dm-dNK/D316N;
Dm-dNK/N318D;
Dm-dNK/N318D/L347P;
Dm-dNK/N321S;
Dm-dNK/F334L;
Dm-dNK/L347P; or
Dm-dNK/K352N (dNK numbering).

19. The enzyme variant of claim 17 being
Bm-dNK/E91D;
Bm-dNK/E91D/N134D;
Bm-dNK/-100D;
Bm-dNK/-100D/N134D;
Bm-dNK/-100D/N134D/K347P;
Bm-dNK/-100D/N134D/L199M/H216S/I217V/D316N;
Bm-dNK/I102T;
Bm-dNK/N114D;
Bm-dNK/N114D/I217V/Y281H;
Bm-dNK/N134D;
Bm-dNK/N134S;
Bm-dNK/N134S/L138S/M139L/K352N;
Bm-dNK/L138S;
Bm-dNK/M139L;
Bm-dNK/M139V;
Bm-dNK/M139V/K347P;
Bm-dNK/V167A;
Bm-dNK/V167A/M171R/A207D;
Bm-dNK/V167S/M171R/A207D;
Bm-dNK/V167A/L199M/H216S/I217V/D316N;
Bm-dNK/V167A/Q321S;
Bm-dNK/V167A/K347P;
Bm-dNK/S168A;
Bm-dNK/S168A/L199M/H216S/I217V/D316N;
Bm-dNK/S168A/N220S;
Bm-dNK/S168A/K347P;
Bm-dNK/L199M;
Bm-dNK/L199M/H216S/I217V/D316N;
Bm-dNK/L199M/V214A/H216S/I217V/D316N;
Bm-dNK/I199M/H216S/I217V/A229S/D316N;
Bm-dNK/M171R/A207D;
Bm-dNK/V214A;
Bm-dNK/H216S;
Bm-dNK/I217V;
Bm-dNK/N220S;
Bm-dNK/T222W;
Bm-dNK/F228C;
Bm-dNK/F228C/V277A/P309R;
Bm-dNK/V277A;
Bm-dNK/A229S;
Bm-dNK/Y281H;
Bm-dNK/P309R;
Bm-dNK/D316N;
Bm-dNK/Q321S;
Bm-dNK/L334L;
Bm-dNK/K347P; or
Bm-dNK/K352N (dNK numbering).

20. The enzyme variant of claim 17 being
Xen-dNK/M51T;
Xen-dNK/M51T/Q168A;
Xen-dNK/G62A;
Xen-dNK/G62A/V167A/E321S;
Xen-dNK/-100D;
Xen-dNK/-100D/N134D;
Xen-dNK/-100D/N134D/E318D;
Xen-dNK/-100D/N134D/N216S/217V;
Xen-dNK/L102T;
Xen-dNK/L102T/E318D;
Xen-dNK/N114D;
Xen-dNK/N114D/L217V/Y281H;
Xen-dNK/N134D;
Xen-dNK/N134S;
Xen-dNK/N134S/L138S/M139L;
Xen-dNK/L138S;
Xen-dNK/M139L;
Xen-dNK/M139V;
Xen-dNK/M139V/E318D/;
Xen-dNK/V167A;
Xen-dNK/V167A/M171R/A207D;
Xen-dNK/V167S/M171R/A207D;
Xen-dNK/V167A/N216S/L217V;
Xen-dNK/V167A/E318D;
Xen-dNK/Q168A;
Xen-dNK/Q168A/N216S/L217V;
Xen-dNK/Q168A/E318D;
Xen-dNK/M171R/A207D;
Xen-dNK/V214A;
Xen-dNK/V214A/N216S/L117V;
Xen-dNK/N216S;
Xen-dNK/N216S/L217V;
Xen-dNK/N216S/217V/A229S;
Xen-dNK/L217V;
Xen-dNK/K222W;
Xen-dNK/Y228C;
Xen-dNK/Y228C/I277A/P309R;
Xen-dNK/A229S;
Xen-dNK/I277A;
Xen-dNK/Y281H;
Xen-dNK/P309R;
Xen-dNK/E318D; or
Xen-dNK/E321S (dNK numbering).

21. The enzyme variant of claim 16, being a hybrid enzyme derived from a Drosophila melanogaster deoxyribonucleoside kinase, and/or a Bombyx mori deoxyribonucleoside kinase, and/or a Xenopus laevis deoxyribonucleoside kinase, and/or an Anopheles gambia deoxyribonucleoside kinase.

22. The enzyme variant of claim 21, which is derived from a Drosophila melanogaster deoxyribonucleoside kinase and a Bombyx mori deoxyribonucleoside kinase, and which comprises the amino acid sequence presented as SEQ ID NO: 10.

23. The enzyme variant of claim 21, which is derived from a Drosophila melanogaster deoxyribonucleoside kinase and a Bombyx mori deoxyribonucleoside kinase, and which comprises the amino acid sequence presented as SEQ ID NO: 12.

24. A vector construct comprising the polynucleotide according to any of claims 1-3.

25. The vector according to claim 24 being a viral vector, in particular a herpes simplex viral vector, an adenoviral vector, an adenovirus-associated viral vector, or a retroviral vector.

26. A packaging cell line capable of producing an infective virion comprising the vector of claim 25.

27. A host cell carrying the mutated polynucleotide according to any of claims 1-3, or the vector according to either of claims 24-25.

28. The cell according to claim 27, which is a human cell, a dog cell, a monkey cell, a rat cell or a mouse cell.

29. A method of sensitising a cell to a prodrug, which method comprises the steps of
(i) transfecting said cell with a polynucleotide sequence according to any of claims 1-3 encoding an enzyme that promotes the conversion of said prodrug into a (cytotoxic) drug; and
(ii) delivering said prodrug to said cell;
wherein said cell is more sensitive to said (cytotoxic) drug than to said prodrug.

30. The method according to claim 29, wherein the prodrug is a nucleoside analogue.

31. The method according to claim 30, wherein the nucleoside analogue is aciclovir (9-[2-hydroxy-ethoxy]-methyl-guanosine), buciclovir, famciclovir, ganciclovir (9-[2-hydroxy-1-(hydroxymethyl)ethoxyl-methyl]-guanosine), penciclovir, valciclovir, trifluorothymidine, AZT (3′-azido-3′-thymidine), AIU (5′-iodo-5′-amino-2′,5′-dideoxyuridine), ara-A (adenosine-arabinoside; Vivarabine), ara-C (cytidine-arabinoside), ara-G (9-beta-D-arabinofuranosylguanine), ara-T, 1-beta-D-arabinofuranosyl thymine, 5-ethyl-2′-deoxyuridine, 5-iodo-5′-amino-2,5′-dideoxyuridine, 1-[2-deoxy-2-fluoro-beta-D-arabino furanosyl]-5-iodouracil, idoxuridine (5-iodo-2′deoxyuridine), fludarabine (2-Fluoroadenine 9-beta-D-Arabinofuranoside), gencitabine, 2′,3′-dideoxyinosine (ddI), 2′,3′-dideoxycytidine (ddC), 2′,3′-dideoxythymidine (ddT), 2,3′-dideoxyadenosine (ddA), 2′,3′-dideoxyguanosine (ddG), 2-chloro-2′-deoxyadenosine (2CdA), 5-fluorodeoxyuridine, BVaraU ((E)-5-(2-bromovinyl)-1-beta-D-arabinofuranosyluracil), BVDU (5-bromovinyl-deoxyuridine), FIAU (1-(2-deoxy-2-fluoro-beta-D-arabinofuranosyl)-5-iodouracil), 3TC (2′-deoxy-3′-thiacytidine), dFdC gemcitabine (2′,2′-difluorodeoxycytidine), dFdG (2′,2′-difluorodeoxyguanosine), or d4T (2′,3′didehydro-3′-deoxythymidine).

32. A method of inhibiting a pathogenic agent in a warm-blooded animal, which method comprises administering to said animal a mutated polynucleotide according to any of claims 1-3, or a vector according to either of claims 24-25.

33. The method according to claim 32, wherein said polynucleotide sequence or said vector is administered in vivo.

34. The method according to either of claims 32-33, wherein said pathogenic agent is a virus, a bacteria or a parasite.

35. The method according to either of claims 32-33, wherein said pathogenic agent is a tumour cell.

36. The method according to either of claims 32-33, wherein said pathogenic agent is an autoreactive immune cell.

37. The method according to any of claims 31-35, further comprising the step of administering a nucleoside analogue to said warm-blooded animal.

38. The method according to claim 37, wherein said nucleoside analogue is aciclovir (9-[2-hydroxy-ethoxy]-methyl-guanosine), buciclovir, famciclovir, ganciclovir (9-[2-hydroxy-1-(hydroxymethyl)ethoxyl-methyl]-guanosine), penciclovir, valciclovir, trifluorothymidine, AZT (3′-azido-3′-thymidine), AIU (5′-iodo-5′-amino-2′,5′-dideoxyuridine), ara-A (adenosine-arabinoside; Vivarabine), ara-C (cytidine-arabinoside), ara-G (9-beta-D-arabinofuranosylguanine), ara-T, 1-beta-D-arabinofuranosyl thymine, 5-ethyl-2′-deoxyuridine, 5-iodo-5′-amino-2,5′-dideoxyuridine, 1-[2-deoxy-2-fluoro-beta-D-arabino furanosyl]-5-iodouracil, idoxuridine (5-iodo-2′deoxyuridine), fludarabine (2-Fluoroadenine 9-beta-D-Arabinofuranoside), gencitabine, 2′,3′-dideoxyinosine (ddI), 2′,3′-dideoxycytidine (ddC), 2′,3′-dideoxythymidine (ddT), 2′,3′-dideoxyadenosine (ddA), 2′,3′-dideoxyguanosine (ddG), 2-chloro-2′-deoxyadenosine (2CdA), 5-fluorodeoxyuridine, BVaraU ((E)-5-(2-bromovinyl)-1-beta-D-arabinofuranosyluracil), BVDU (5-bromovinyl-deoxyuridine), FIAU (1-(2-deoxy-2-fluoro-beta-D-arabinofuranosyl)-5-iodouracil), 3TC (2′-deoxy-3′-thiacytidine), dFdC gemcitabine (2′,2′-difluorodeoxycytidine), dFdG (2′,2′-difluorodeoxyguanosine), or d4T (2′,3′didehydro-3′-deoxythymidine).

39. A pharmaceutical composition comprising a mutated polynucleotide according to any of claims 1-3, or a vector according to either of claims 24-25.

40. A pharmaceutical composition comprising the enzyme variant according to any of claims 4-23, and a pharmaceutically acceptable carder or diluent.
